(19) United States
(12) Patent Application Publication
(10) Pub. No.: US 2025/0064333 A1
(43) Pub. Date: Feb. 27, 2025

(54) Normalization and Calibration for Devices and Systems for Measuring Peripheral Hemodynamics

(71) Applicant: Washington University, St. Louis, MO (US)

(72) Inventors: Christine O'Brien, St. Louis, MO (US); Leonid Shmuylovich, St. Louis, MO (US)

(21) Appl. No.: 18/812,459

(22) Filed: Aug. 22, 2024

Related U.S. Application Data
(60) Provisional application No. 63/578,082, filed on Aug. 22, 2023.

(51) Int. Cl. A61B 5/026; A61B 5/00; G16H 50/30

(52) U.S. Cl. CPC A61B 5/026; A61B 5/0053; A61B 5/7264; A61B 5/7271; G16H 50/30; A61B 2560/0462

(57) ABSTRACT

A method for monitoring peripheral blood flow of a patient using a wearable device includes monitoring peripheral blood flow of the patient under a first physiologic condition and monitoring peripheral blood flow of the patient under a second physiologic condition different than the first physiologic condition. A dimensionless relative index of the monitored peripheral blood flow of the patient i) under the second physiologic condition based on the monitored peripheral blood flow under the first physiologic condition, or ii) under the first physiologic condition based on the monitored peripheral blood flow under the second physiologic condition is generated. An indication of the dimensionless relative index is output.

### CROSS REFERENCE TO RELATED APPLICATIONS

This application claims priority to U.S. provisional application No. 63/578,082 filed on Aug. 22, 2023, which is hereby incorporated by reference in its entirety.

### STATEMENT REGARDING FEDERALLY SPONSORED RESEARCH & DEVELOPMENT

This invention was made with government support under HD103954 awarded by the National Institutes of Health. The government has certain rights in the invention.

### BACKGROUND

The present disclosure generally relates to systems, devices, and methods for non-invasively measuring peripheral hemodynamics in a subject. More particularly, this disclosure relates to normalization and calibration for such systems, devices, and methods.

Postpartum hemorrhage (PPH), defined as the loss of 1 L of blood or more within 24 hours after birth, is the leading cause of maternal mortality worldwide with an estimated 14 million cases each year resulting in 130,000 deaths. Importantly, PPH has been noted as the most preventable cause of maternal mortality. The leading factors causing preventable PPH are delays in diagnosis and treatment. PPH prevention is especially critical in low resource settings that often have low blood stores for transfusion and consequently rely primarily on early pharmacologic treatment for PPH. The United States (US) has the highest maternal mortality rate of any developed country, where the most commonly used method for PPH diagnosis is a visual estimation of blood loss, a method known to underestimate blood loss. There is a need for an early and accurate PPH alert system.

Monitoring peripheral hemodynamics may be useful for detection of PPH and other blood flow and blood loss conditions. Known devices for monitoring peripheral blood flow however, typically provide relative measurements that make clinically meaningful interpretation difficult, especially when a “healthy” baseline cannot be obtained. Moreover, many such devices are affected by skin color, potentially resulting in different measurements under identical physiological conditions depending on the skin color of the patient. There is a need for devices that provide subject-specific perfusion range that can be determined regardless of whether a healthy baseline measurement can be acquired.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

According to one aspect of the present disclosure, a method for monitoring peripheral blood flow of a patient using a wearable device includes monitoring peripheral blood flow of the patient under a first physiologic condition and monitoring peripheral blood flow of the patient under a second physiologic condition different than the first physiologic condition. A dimensionless relative index of the monitored peripheral blood flow of the patient i) under the second physiologic condition based on the monitored peripheral blood flow under the first physiologic condition, or ii) under the first physiologic condition based on the monitored peripheral blood flow under the second physiologic condition is generated. An indication of the dimensionless relative index is output.

Another aspect of the present disclosure is a system for monitoring peripheral blood flow of a patient. The system includes a housing configured for wearable attachment to the patient, a physiologic sensor for monitoring peripheral blood flow of the patient disposed within the housing, a processor communicably couplable to the physiologic sensor, and a memory communicably coupled to the processor. The memory stores instructions that, when executed by the processor, program the processor to monitor peripheral blood flow of the patient under a first physiologic condition using the physiologic sensor, monitor peripheral blood flow of the patient under a second physiologic condition different than the first physiologic condition using the physiologic sensor, generate a dimensionless relative index of the monitored peripheral blood flow of the patient under the second physiologic condition based on the monitored peripheral blood flow under the first physiologic condition or under the first physiologic condition based on the monitored peripheral blood flow under the second physiologic condition, and output an indication of the dimensionless relative index.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic diagram illustrating the acquisition of a laser speckle flow index (LSFI) image from a human finger.

FIG. 1B contains laser speckle flow index (LSFI) images of a middle and ring finger with the left side (left) or right side (right) wrapped with a band.

FIG. 2A is a spectroscopic image measuring absorption between 880-1700 nm of swine whole blood diluted by 10% and 20% with phosphate-buffered saline (PBS) control.

FIG. 2B is the ratio of 1020 to 1350 nm absorption in whole and diluted swine blood along with PBS control, which revealed significant differences between groups.

FIG. 3 is a schematic illustration of the measurement of a laser speckle flow index from a swine wrist using source-detector offset to monitor skin and muscle perfusion during hemorrhage.

FIG. 4 is a photo of a wearable laser speckle sensor in one aspect placed on a swine wrist and used during swine hemorrhage experiment.

FIG. 5A shows short wave infrared spectra of varying concentrations of hemoglobin in saline.

FIG. 5B is the ratio of 1020:1350 nm vs. hemoglobin concentration taken from the spectra in FIG. 5A.

FIG. 6 contains CAD renderings and pictures of a wearable hemodilution sensor in one aspect.

FIG. 7A is a graph showing swine hemorrhage blood loss before and after a crystalloid infusion protocol. Between 90 and 115 minutes the laser speckle sensor was misaligned resulting in spurious signal (gray).

FIG. 7B is a graph showing Laser speckle flow index (LSFI) results from the swine hemorrhage study illustrated in FIG. 7A showing a correlation with blood withdrawal and crystalloid infusion. Between 90 and 115 minutes the laser speckle sensor was misaligned resulting in spurious signal (gray).

FIG. 7C is a graph showing the normalized LSFI results throughout the hemorrhage study of FIG. 7A. Between 90 and 115 minutes the laser speckle sensor was misaligned resulting in spurious signal (gray).

FIG. 8A contains a first part of a series of graphs showing LSFI vs time, calculated at 10 and 100 frames per second, using either a ones square matrix convolution (top row) or an identity matrix convolution (bottom row), and using full image size, half image size, or quarter image size cropping (encompassing 12 distinct convolutions).

FIG. 8B contains a second part of the series of graphs from FIG. 8A. FIGS. 8A and 8B are collectively referred to herein as FIG. 8.

FIG. 9A is a graph of swine hemorrhage blood loss before and after a crystalloid infusion protocol.

FIG. 9B is a graph showing swine hemorrhage blood loss before and after a crystalloid infusion protocol.

FIG. 9C is a graph showing an unsmoothed laser speckle flow index (LSFI) resulting from the swine hemorrhage study of FIG. 9A, showing a correlation with blood withdrawal and crystalloid infusion.

FIG. 9D is a graph showing a smoothed laser speckle flow index (LSFI) result from the swine hemorrhage study in FIG. 9C, showing a correlation with blood withdrawal and crystalloid infusion.

FIG. 9E is a graph showing pre-vein collapse blood loss vs. LSFI from unsmoothed LSFI data.

FIG. 9F is a graph showing pre-vein collapse blood loss vs. LSFI from smoothed LSFI data.

FIG. 9G is a graph showing post-vein collapse blood loss vs. LSFI from unsmoothed LSFI data.

FIG. 9H is a graph showing post-vein collapse blood loss vs. LSFI from smoothed LSFI data.

FIG. 9I is a graph showing crystalloid infusion vs. LSFI from unsmoothed LSFI data.

FIG. 9J is a graph showing crystalloid infusion vs. LSFI from smoothed LSFI data.

FIG. 10A contains a first part of a series of graphs showing systolic blood pressure (SBP), diastolic blood pressure (DBP), pulse pressure (SBP-DBP), temperature, heart rate (HR), and respiratory rate over time during a swine hemorrhage study (top row), corresponding normalized vital signs (middle row), and blood volume (bottom row). With the exception of temperature, vital signs parameters demonstrated a decrease with blood loss (blue), and an increase with crystalloid administration (red).

FIG. 10B contains a second part of the series of graphs from FIG. 10A FIGS. 10A and 10B are collectively referred to herein as FIG. 10.

FIG. 11A contains a first part of a series of graphs that correlate normalized vital signs and blood loss. Top panel includes all time points, and the bottom panel includes times preceding vein collapse.

FIG. 11B contains a second part of the series of graphs from FIG. 11A. FIGS. 11A and 11B are collectively referred to herein as FIG. 11.

FIG. 12 is a correlation heat map for LSFI and vital signs during swine blood loss.

FIG. 13 is a graph showing speckle plethysmography (SPG) and photoplethysmography (PPG) traces from a swine hemorrhage study. The SPG signal has higher signal to noise ratio (SNR) compared to PPG, although both waveforms are data rich. The time delay (At) between peaks from SPG and PPG has been shown to correspond with systemic vascular resistance, and can be easily calculated using the disclosed wearable laser speckle sensor.

FIG. 14 contains CAD renderings of a completely wearable laser speckle sensor in one aspect.

FIG. 15 is a block diagram of a system for monitoring peripheral blood flow of a patient incorporated in a wearable device.

FIG. 16 is a block diagram of a system for monitoring peripheral blood flow of a patient including a wearable device and a remote device.

FIG. 17 is a flow diagram of an example method for monitoring peripheral blood flow of a patient.

FIG. 18 illustrates an example experiment testing the example methods of monitoring peripheral blood flow using pneumatic cuff occlusion and fixed volume hemorrhage in swine.

FIG. 19 representative trace of blood flow sensor data from one human participant in an experiment.

FIG. 20A is a graph of changes in blood flow signals during exercise for two groups of human patients.

FIG. 20B is a graph of changes in blood flow signals during exercise for two groups of human patients from FIG. 20A normalized according to the present disclosure.

FIG. 21 illustrates an occlusion normalization procedure according to the present disclosure, including the identification of patient-specific “danger zones.”

FIG. 22 is a graph of occlusion normalized blood flow data collected during another experiment using adult humans.

FIG. 23A is a side-view of a tissue mimicking flow phantom with a thin pigmented layer on top.

FIG. 23B is a cross-section view of the tissue mimicking flow phantom of FIG. 23A with a hollow channel through which fluid may be flowed clearly visualized beneath thin pigmented top layer.

FIG. 24A is a graph of raw blood flow data from a wearable device according to the present disclosure when measuring simulated blood flow through phantoms according to FIGS. 23A and 23B with two different levels of simulated skin pigmentation.

FIG. 24B is a graph of the raw data of FIG. 24A normalized according to a first technique.

FIG. 24C is a graph of the raw data of FIG. 24A normalized according to a second technique.

FIG. 24D is a graph of the raw data of FIG. 24A normalized according to a third technique.

FIG. 25 is a graph of occlusion normalized monitored blood flow data and measured blood volume from an experiment on a female swine.

Corresponding reference characters indicate corresponding parts throughout the drawings. There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, systems, devices, and methods for non-invasively monitoring peripheral hemodynamics in a subject are disclosed herein. More particularly, this disclosure relates to normalization and calibration for such systems, devices, and methods. Various aspects may include monitoring a subject for hemorrhage including, but not limited to post-partum hemorrhage (PPH).

During hemorrhage, two important compensatory mechanisms occur: 1) blood is shunted from the periphery to vital organs by constricting peripheral vessels; 2) interstitial fluid is transferred into vessels to maintain blood volume, effectively reducing hemoglobin (Hb) concentration and hematocrit (Hct). These compensatory mechanisms help stabilize the patient and delay the time until global vascular indicators such as blood pressure and heart rate are affected. Thus, the monitoring of peripheral blood flow and blood content can detect relatively minor decreases in Hb and Hct that serve as early indicators of hemorrhage.

Optical technologies are well suited to noninvasively measure blood flow and blood content. Laser speckle imaging directly measures flowing blood cells and the laser speckle flow index (LSFI) is proportional to velocity. Optical spectroscopy provides quantification of blood and tissue oxygenation (near-infrared region), as well as quantification of water (infrared region), enabling observation of water transfer to the vasculature during hemorrhage. Optical monitoring techniques are non-ionizing, label-free, fast, and can be implemented using small and wearable devices to provide a continuous ergonomic sensing system. Preliminary experiments described in the Examples below demonstrate sensitivity to reduced perfusion in vivo using laser speckle imaging, and/or optical spectroscopy measures significant differences between blood samples diluted with saline to physiologic levels seen in PPH.

Optical monitoring of blood flow and blood content has numerous advantages: sensitivity to multiple intrinsic biological chromophores (melanin, deoxy- and oxyhemoglobin, lipids, proteins, and water) depending upon the optical wavelengths used; ability to detect and quantify blood flow; high potential for small, simple, and wearable hardware; and rapid results. Such characteristics are ideal for patient monitoring, as evidenced by the pulse oximeter, an optical device used globally for patient monitoring. Optical spectroscopy-based tools have been developed for in vitro and in vivo measurement of hemoglobin, and continuous noninvasive optical spectroscopy tools have been used extensively in critical care patients to monitor changes in Hb concentration caused by hypovolemia. Although the perfusion index is known to be skewed and has high patient variability, previous results are encouraging and show that non-invasive optical measures can detect early signs of postpartum blood loss.

In various aspects, a multifunctional sensing system to track Hb concentration and peripheral perfusion for the monitoring and/or early detection of postpartum hemorrhage (PPH) is disclosed. The disclosed system includes an LSFI (laser speckle flow index) sensor and/or a multispectral Hb sensor. In various aspects, the disclosed system synergistically combines laser speckle imaging for blood perfusion measurements and near infrared/short wave infrared (NIR/SWIR) spectroscopy for monitoring Hb, to provide tracking of the two separate and independent compensatory mechanisms of PPH. Other embodiments include only the laser speckle imaging or only the NIR/SWIR spectroscopy.

The LSFI (laser speckle flow index) sensor uses laser speckle imaging of peripheral skin and muscle tissues to monitor peripheral perfusion. The laser speckle sensor performs peripheral perfusion monitoring which is proportional to blood flow velocity to provide a more direct measure of perfusion than the perfusion index. In one aspect, the LSFI sensor includes a 785 nm laser diode and a video camera to obtain laser speckle contrast images. The laser speckle contrast images are processed using established algorithms to obtain laser speckle flow index images indicative of peripheral perfusion. In some aspects, the LSFI sensor is a wearable LSFI (laser speckle flow index) sensor positioned over a muscle and gently held in place with a band, ensuring placement over muscles in order to measure both skin and muscle blood flow.

The disclosed multispectral Hb sensor of the disclosed system uses NIR absorption of Hb and SWIR absorption of water; absorption by water in the SWIR range is stronger than within the spectral ranges used by existing devices, thereby improving sensitivity and specificity of water measurements obtained using the disclosed system. In some aspects, the multispectral Hb sensor of the disclosed system includes two light-emitting diodes (LEDs) at different wavelengths that are used to monitor blood content. In one aspect, an 800 nm LED (L1) is used for Hb measurement, as this is the point where oxy- and deoxyhemoglobin absorb at the same rate, eliminating variability caused by the oxygen saturation of Hb to focus solely on total hemoglobin. For water measurements, a 1340 nm LED (L2) is used to balance between high contrast and subcutaneous penetration depth. The multispectral Hb sensor further includes two photodiodes for detecting LED light: one sensitive to NIR Hb signal (D1, silicon detector), and one sensitive to SWIR water signal (D2, InGaAs detector). Ratiometric calculations and computational removal of room light contamination are performed using algorithms similar to those used in pulse oximeters. In some aspects, the multispectral Hb sensor may further include a microprocessor to perform de-noising and ratiometric calculations throughout data capture. For pulsatile blood flow, ratiometric calculations and removal of room lighting are performed using algorithms similar to those used in standard pulse oximetry to extract the Hb to water ratio.

In various aspects, data obtained using the sensors of the disclosed multispectral Hb sensor are transferred to secure cloud storage using Bluetooth Low Energy (BLE) wireless network.

Additional description of the disclosed system, devices, and methods are provided below.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

To develop and validate a post-partem (PPH) hemorrhage monitoring system, the following experiments were conducted.

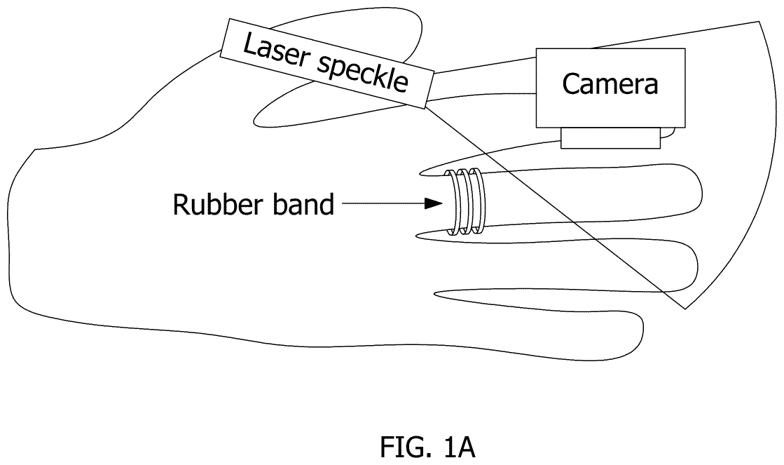

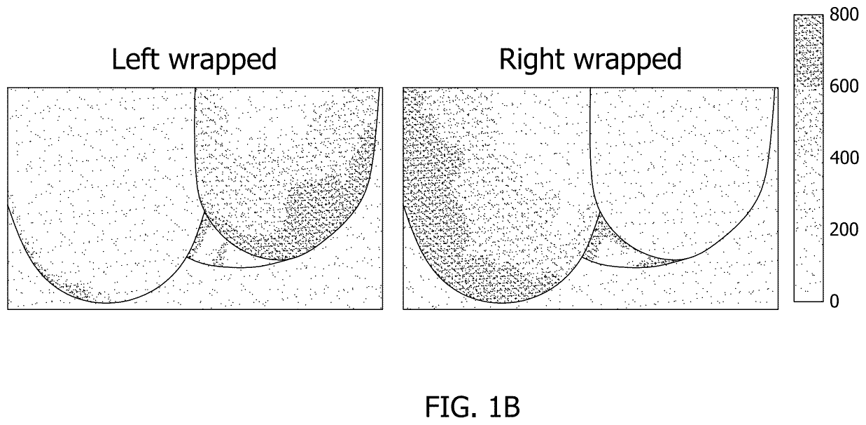

A laser speckle imaging system using a 785 nm laser diode and video camera was assembled as illustrated in FIG. 1A. To test the ability of the system to measure reduced blood flow, the middle finger of a volunteer was wrapped tightly with a rubber band to disrupt blood flow and then the wrapped middle finger and the adjacent unwrapped ring finger were measured using the camera (FIG. 1A). This experiment was repeated with the ring finger wrapped and the middle finger unwrapped. Laser speckle contrast images were processed using established algorithms and the resulting laser speckle flow index images are shown in FIG. 1B.

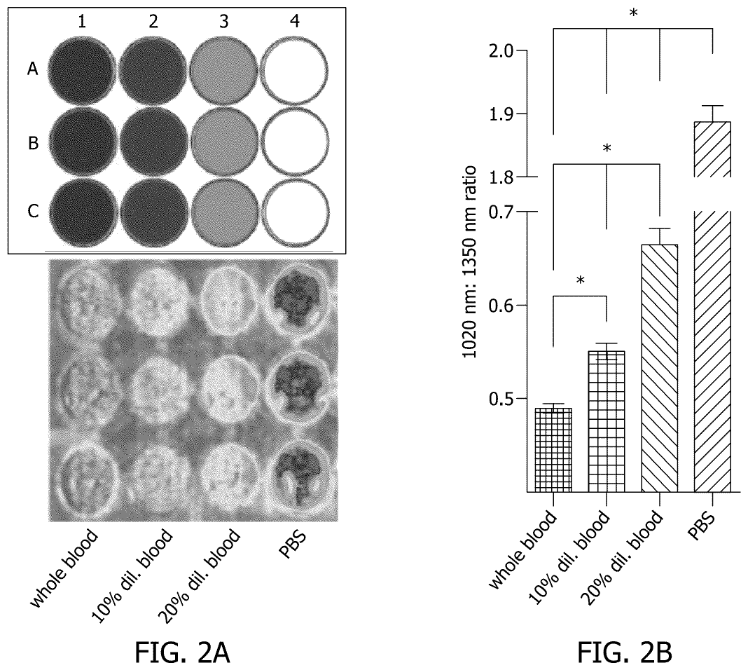

The ability of SWIR spectroscopy using a hyperspectral imaging system to distinguish varying levels of Hb was tested using swine blood diluted with PBS. Wells of whole blood, 10% diluted blood, 20% diluted blood, and PBS were measured in triplicate (FIG. 2A). T hyperspectral imaging system was used to image absorption at wavelengths spanning 880-1700 nm. The ratio of the image at 1020 nm and 1350 nm was calculated and is shown in FIG. 2A. The intensity ratios in each well were quantified and the mean and standard deviation of the triplicate measures are summarized in FIG. 2B. Statistically significant increases were observed across every group as measured by ANOVA with Bonferroni's multiple comparison test, demonstrating efficacy of this technique for quantifying differences in Hb concentration as measured via water to Hb ratio (1020 nm:1330 nm).

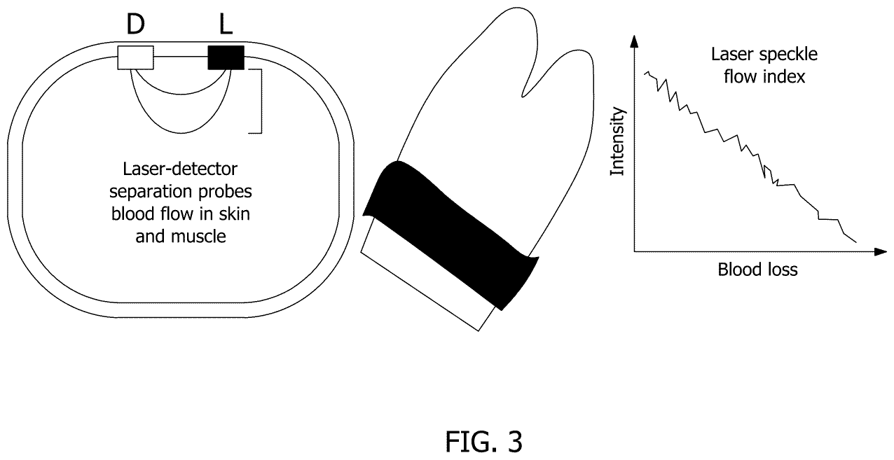

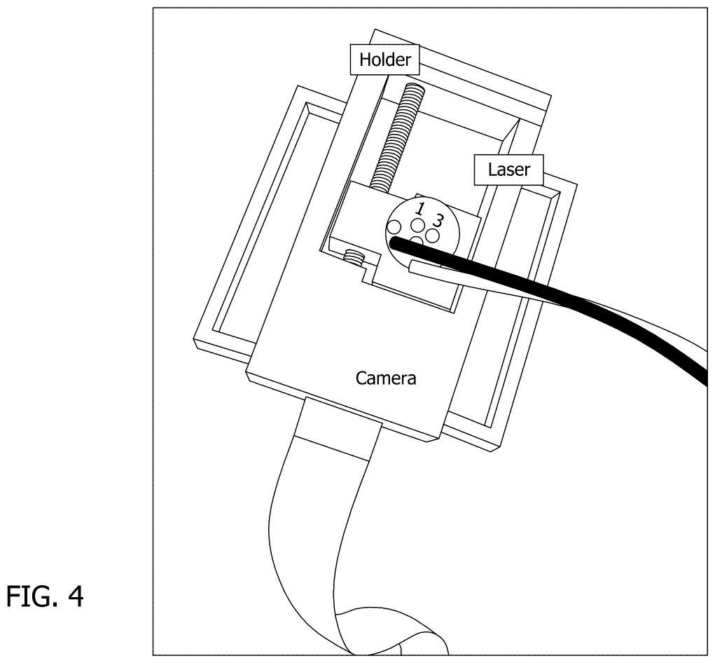

The laser speckle sensor is an optical sensor designed in reflectance mode, i.e. the light source and detector are positioned on the same side, however a transmission mode design could also be used. The reflection mode device, illustrated in FIG. 4, includes of a 50 mW 780 nm laser module (Laserland, 11071013) and a double-lens Raspberry Pi camera sensor (Raspberry Pi is a trademark of Raspberry Pi Ltd.). The double lens camera sensor consists of a Raspberry Pi Camera Module V2, with the lens turned to have a maximal focal distance, and a second lens, from a second V2 module, inverted and attached directly to the surface of the first lens. A 0.4 mm sapphire window (Edmund Optics, 43-628) is attached to the second lens, allowing for the sensor to be in focus on objects sitting or pressing directly on the window. The laser and double-lens camera sensor are held in position to be directly in contact with a subject by a 3D printed holder. The 3D printed holder is adjustable and allows for the laser and sensor distance to be varied to a specified distance and then fixed, for optimization of signal intensity and contrast. The laser module is powered by a 3.3 V wall source, but can be powered by a battery. The camera sensor is powered and controlled by a Raspberry Pi 4 Model 4 computer board. A schematic and photo of this device is shown in FIGS. 3 and 4, respectively.

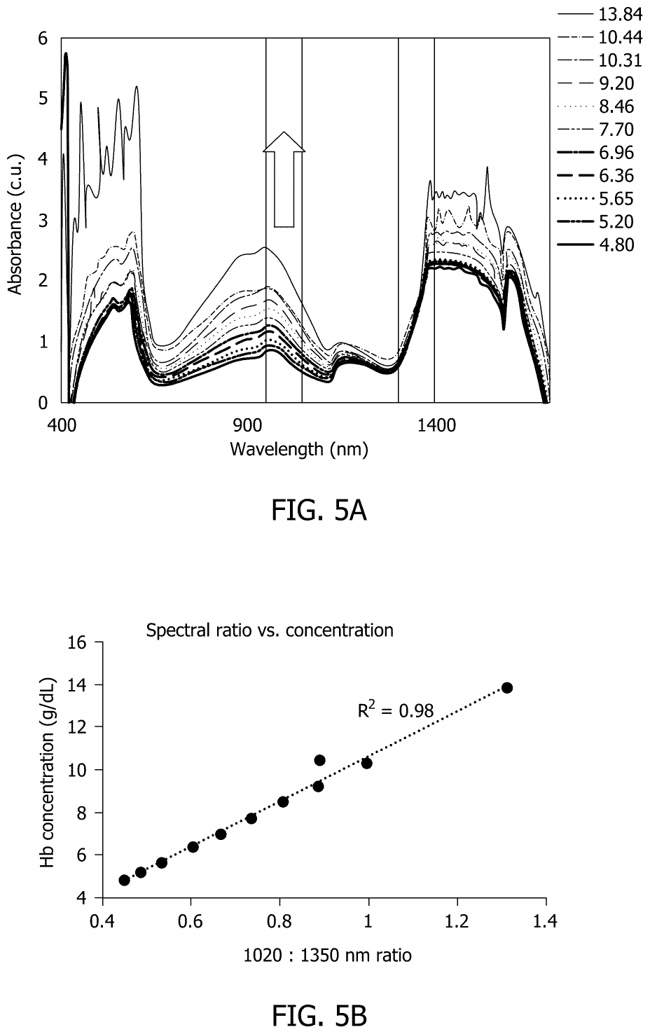

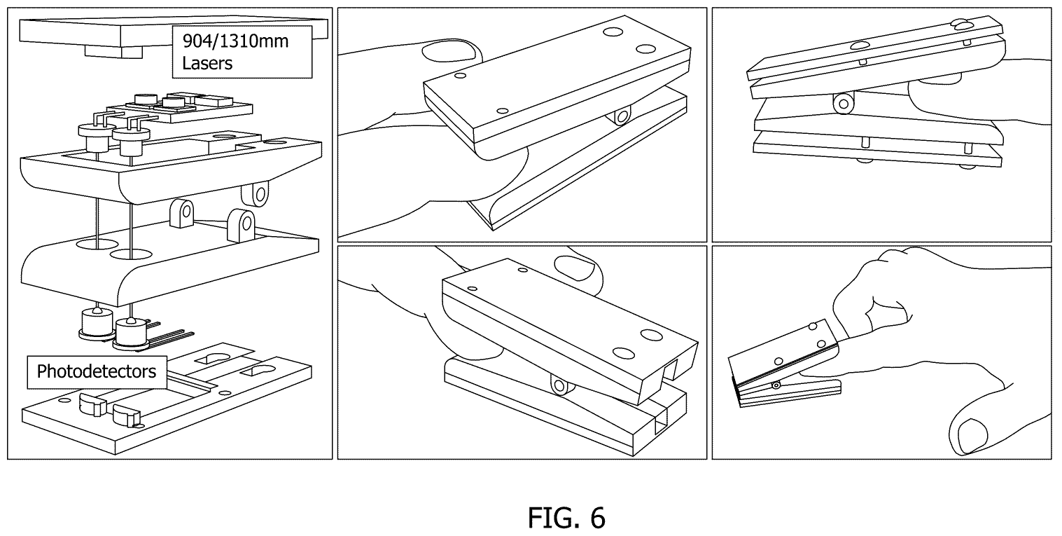

The wavelengths chosen for use in the hemodilution sensor were based on preliminary data using a short wave infrared spectrometer that measured spectral changes across hemoglobin samples ranging in concentration from 4.8-13.84 g/dL (FIG. 5A). The results revealed significant increases across 850-1000 (attributed to hemoglobin) and a constant response between 1300-1350 (attributed to water) across the concentrations. The ratio of intensities at two wavelengths in these bands, 1020 nm and 1350 nm, were plotted against hemoglobin concentration (FIG. 5B) and an R2=0.98 was achieved; lasers from these bands were chosen for use in the wearable hemodilution sensor design. The hemodilution sensor (FIG. 6) consists of two InGaAs photodiode detectors (although could be completed with one) with sensitivity from 800-1700 nm (Thorlabs, FGA01), directly across from a 904 nm laser (Thorlabs, L904P010) and a 1310 nm laser (Thorlabs, ML725B8F) which were each connected to a laser driver to maintain constant current and therefore constant optical output (IC Haus, WK2D) (FIG. 6). The two light sources were modulated at 25 Hz in alternating 20 ms intervals using digital pins from an Arduino Uno, and chosen to detect relative changes in hemoglobin and water. The photodiode detected the transmitted optical signals via an analog pin on the Arduino Uno, which has a 10 bit ADC. Arduino code controlled the light source modulation and separated the 900 nm and 1310 nm signals into distinct channels and plotted their output in real time. The Arduino was plugged into a laptop computer via USB/USB B connection. The photodiode bias voltage was supplied using an external wall mounted power supply of 12 V, but could be powered by a battery. Although the current design is in transmission mode, it could be built in reflectance mode.

A twelve week old male White Yorkshire x Landrace pig was anesthetized and cut downs were performed on the femoral vein and artery to establish a blood withdrawal port and to insert an arterial blood pressure catheter, respectively. The estimated blood volume (EBV, 58-74 mL/kg) was calculated (2100-2500 mL) based on the swine weight (34.5 kg). Once the catheter was in place, the hemodilution sensor (FIG. 6) was placed on the ear of the pig and the laser speckle sensor was placed on the left posterior hock after removing hair with an electrical hair trimmer (FIG. 3). The sensors recorded baseline levels for 15 minutes, after which we removed 1.5% blood volume (33 mL) every 5 minutes, followed by a 2 mL saline flush. This continued for a total of 800 mL blood removed over the course of 2.5 hours, which was an estimated blood loss of ˜31-38%. In parallel, heart rate, systolic and diastolic blood pressure, temperature, blood oxygen saturation, respiratory rate and hematocrit were measured every 15 minutes throughout the procedure. After reaching 800 mL of blood loss, the swine was then reinfused every 5 minutes with 33 mL of crystalloids for a total of 264 mL over 35 minutes.

Data was collected using a Python script from the Raspberry Pi. Data was collected via video for 10 seconds every minute from 15 minutes before the start of the blood loss protocol, until 5 minutes after the final crystalloid infusion. Video data was saved directly onto the Raspberry Pi hard drive. Data was processed post-study as a rolling average of the speckle index over time. The camera was set to capture video at 100 frames per second, with a 5 ms exposure time.

In laser speckle contrast imaging, contrast is generated by applying a spatial averaging algorithm within a square sliding window that spans a raw speckle image. Specifically, to find the speckle contrast at a given pixel (x,y), one defines a square window centered about (x,y) and divides the standard deviation of the pixel intensity within that window by the mean pixel intensity within the window. For real time processing of video speckle data, this algorithm must be applied to every video frame, and with frame rates up to 100 fps, efficient speckle contrast algorithms are critical. The standard deviation of pixel intensity within each sliding window is related to the variance of the pixel intensity, which is determined by taking the difference between the mean of the square of the raw image pixel intensity and square of the mean of the raw image pixel intensity. An established approach for efficiently determining these rolling averaged images is convolving a square array of ones with both the square of the raw image and with the raw image itself, resulting in the following expression for the speckle contrast image pixel intensity (k):

$$k = \frac{\sqrt{\frac{{n}^{2} {{I}_{s}}^{2} ⊗ ( \begin{matrix}1 & … & 1 \\ ⋮ & 1 & ⋮ \\ 1 & … & 1\end{matrix} ) - {( {I}_{s} ⊗ ( \begin{matrix}1 & … & 1 \\ ⋮ & 1 & ⋮ \\ 1 & … & 1\end{matrix} ) )}^{2}}{{n}^{2} ( {n}^{2} - 1 )}}}{( \frac{1}{{n}^{2}} {I}_{s} ⊗ ( \begin{matrix}1 & … & 1 \\ ⋮ & 1 & ⋮ \\ 1 & … & 1\end{matrix} ) )} \qquad ( Equation 1 )$$

where the sliding windows have dimensions n×n (n is odd without loss of generality), Iₛ is the raw image intensity, and the ones matrices have the same dimension as the sliding window. The disclosed hemorrhage monitoring system captures a video stream, where each frame is a raw intensity image. To detect peripheral vascular flow, each frame is analyzed according to Equation 1 to yield a speckle contrast image k, and then the average pixel intensity <k> across the entire image (excluding a (n−1)/2 thick rectangular border) is calculated and stored for each frame. Thus, the output signal is a single averaged speckle contrast value over time.

For each captured frame, the average speckle contrast <k> can be implemented in python using methods from publicly available software libraries like Numpy.mean( ), Numpy.ones( ) Scipy.signal.convolve2d ( ) or Scipy.signal.fftconvolve( ). However, because the relevant output signal for monitoring is a measure of average speckle contrast and not the speckle contrast image itself, an alternative approach is possible that significantly speeds up processing time. Consider an alternative speckle contrast index k′:

$${k}^{′} = \frac{\sqrt{\frac{{{nI}_{s}}^{2} ⊗ ( \begin{matrix}1 & … & 1 \\ ⋮ & 1 & ⋮ \\ 1 & … & 1\end{matrix} ) - {( {I}_{s} ⊗ ( \begin{matrix}1 & … & 1 \\ ⋮ & 1 & ⋮ \\ 1 & … & 1\end{matrix} ) )}^{2}}{n ( n - 1 )}}}{\frac{1}{n} {I}_{s} ⊗ ( \begin{matrix}1 & … & 1 \\ ⋮ & 1 & ⋮ \\ 1 & … & 1\end{matrix} )} \qquad ( Equation 2 )$$

where the ones matrices in Equation 1 are replaced with n×n identity matrices (n is odd). This speckle contrast index k′ is based on averages of the raw speckle image intensity and square of the raw image intensity along the diagonal of the square window (effectively replacing each pixel (x,y) with the sum of n pixels along a diagonal with (x,y) at the center):

$${I}_{{S}_{( x , y )}} = {\sum }_{- \frac{n - 1}{2}}^{\frac{n - 1}{2}} {I}_{{s}_{( x + k , y + k )}} \qquad ( Equation 3 )$$

For a 7 by 7 square window, this diagonal average can be written directly for the raw image and square of the raw image, without applying convolutions, as:

$${〈 {I}_{S} 〉}^{′} = ( {I}_{s} [ 6 : h - 1 , 6 : w - 1 ] + {I}_{s} [ 5 : h - 2 , 5 : w - 2 ] + {I}_{s} [ 4 : h - 3 , 4 : w - 3 ] + {I}_{s} [ 3 : h - 4 , 3 : w - 4 ] + {I}_{s} [ 2 : h - 5 , 2 : w - 5 ] + {I}_{s} [ 1 : h - 6 , 1 : w - 6 ] + {I}_{s} [ 0 : h - 7 , 0 : w - 7 ] ) \qquad ( Equation 4 )$$

$${〈 {I}_{s}^{2} 〉}^{′} = ( {I}_{s}^{2} [ 6 : h - 1 , 6 : w - 1 ] + {I}_{s}^{2} [ 5 : h - 2 , 5 : w - 2 ] + {I}_{s}^{2} [ 4 : h - 3 , 4 : w - 3 ] + {I}_{s}^{2} [ 3 : h - 4 , 3 : w - 4 ] + {I}_{s}^{2} [ 2 : h - 5 , 2 : w - 5 ] + {I}_{s}^{2} [ 1 : h - 6 , 1 : w - 6 ] + {I}_{s}^{2} [ 0 : h - 7 , 0 : w - 7 ] ) \qquad ( Equation 5 )$$

where the raw image Iₛ dimensions are h by w. Then Equation 2 can be rewritten:

$${k}^{′} = \frac{\sqrt{\frac{7 * {〈 {I}_{S}^{2} 〉}^{′} - {( {〈 {I}_{S} 〉}^{′} )}^{2}}{7 * 6}}}{\frac{1}{7} {〈 {I}_{S} 〉}^{′}} \qquad ( Equation 6 )$$

In testing 100 fps video streams, Equation 6 was found to be 3-5 times faster than Equation 1. Furthermore this hard-coded approach has the added value of simplicity, without the need for 3^{rd} party python libraries like Scipy for implementing efficient convolution.

Further improvements in processing speed can be realized by cropping the video stream data, for example from 640×480 to 320×240, 160×120 or smaller. In addition, the frame rate may be reduced to 30 fps, 10 fps, or smaller. Finally, the video stream that is captured can be captured in YUV mode rather than RGB mode. In YUV mode only the first third of the full frame bytes need to be utilized (the ‘Y’ channel) as this channel contains the pixel intensity, while the ‘U’ and ‘V’ channels contain pixel color. Using RGB video mode by contrast requires reading in 3 times more data per frame followed by conversion to gray scale for each captured frame.

### Results

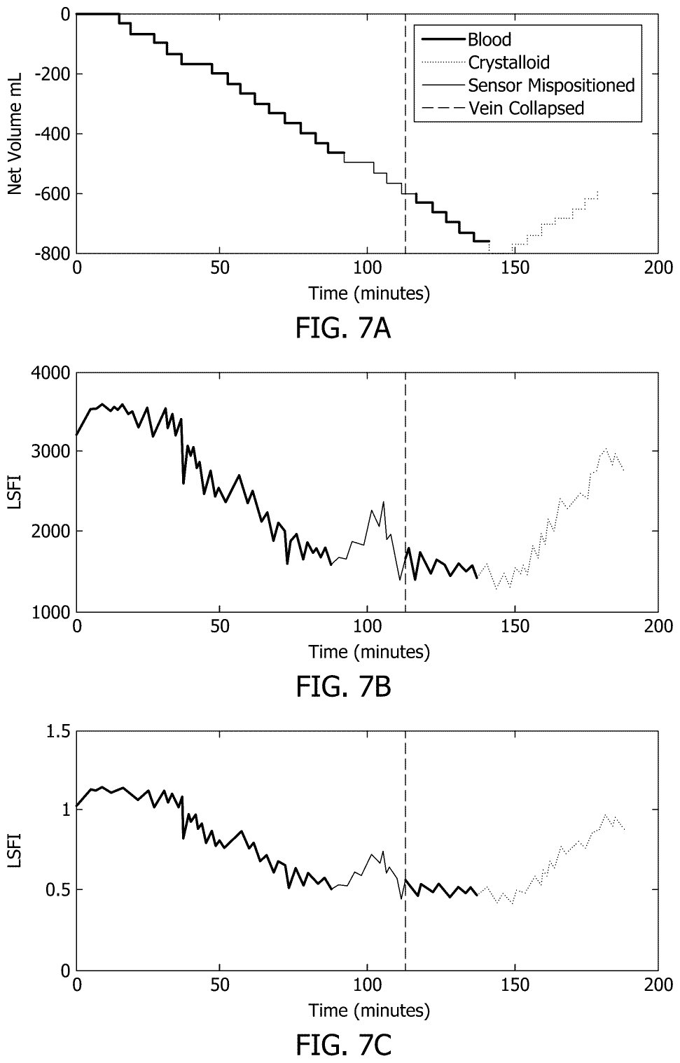

To monitor hemorrhage (and/or postpartum hemorrhage), a laser speckle flow index (LSFI) can be derived from the inverse square of the speckle contrast index:

$$LSFI = \frac{1}{〈 {( k )}^{2} 〉} \qquad ( Equation 7 )$$

where <k> is an averaged speckle contrast index. This average can be defined different ways. The average speckle contrast <k> can be defined at a given time point as the average value across all pixels in a processed speckle video frame captured at that time point. One can also average across multiple frames, obtaining the average speckle contrast index over a 10 second time period, for example. We have tested the ability of LSFI to noninvasively detect hemorrhage-induced peripheral vasoconstriction due to physiologic compensatory mechanisms in a swine experimental model. After obtaining femoral vein access, nearly 800 mL of blood (˜30% blood volume) was removed over a duration of 2 hours (33 mL blood every 5 minutes). Subsequently 231 mL of crystalloid was administered over a duration of 35 minutes (33 mL crystalloid every 5 minutes) (FIG. 7). Of note, veins collapsed at 113 minutes from the start of the experiment (dotted vertical line).

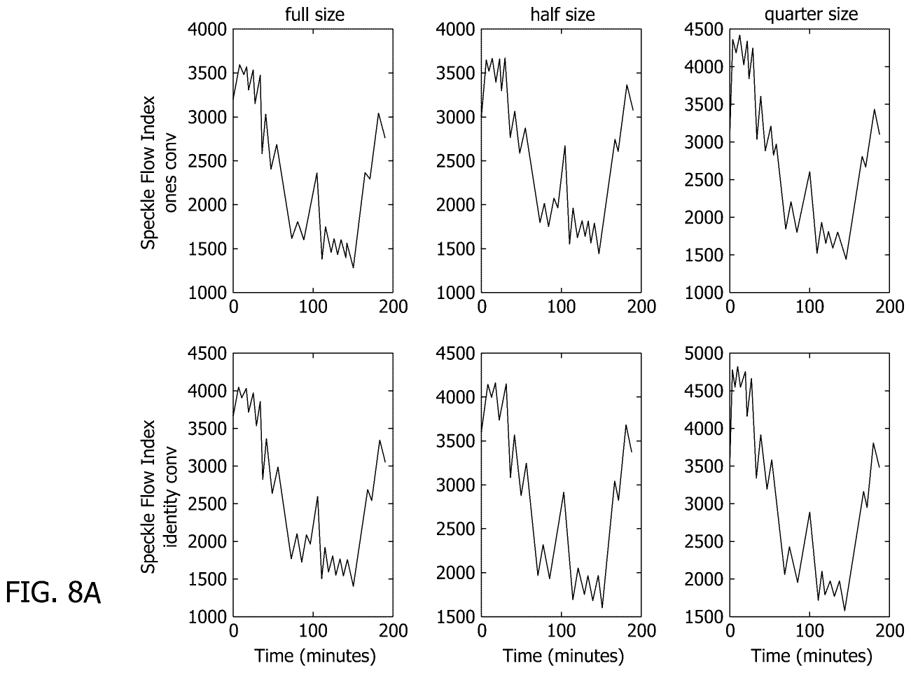

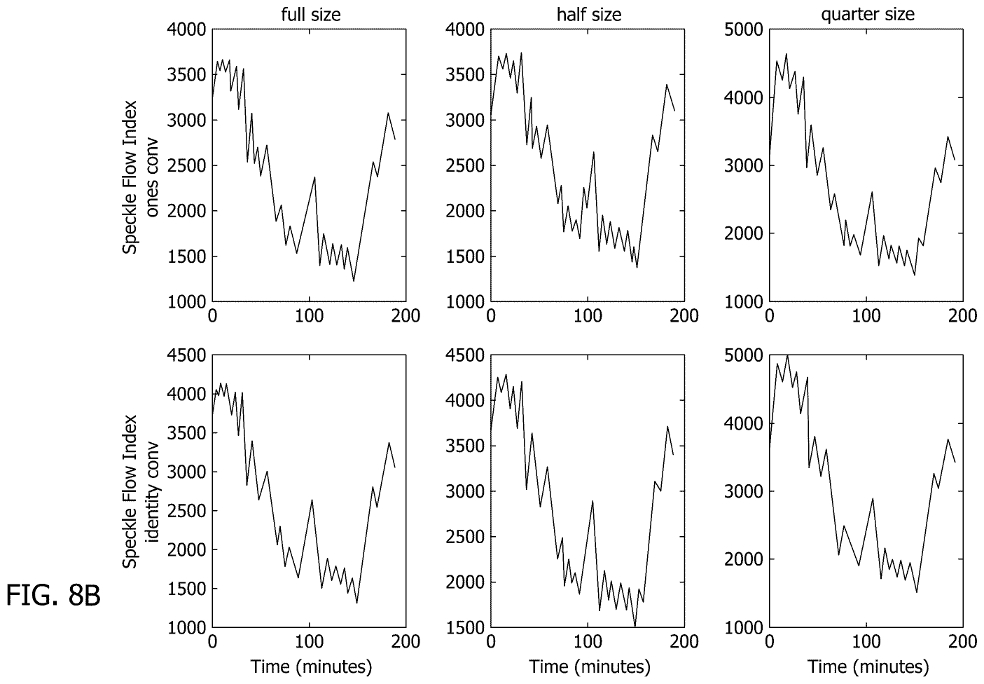

The hemorrhage monitor system was worn on the swine wrist, and 10 s of 320×240 100 fps speckle video was recorded every minute throughout the experiment. For each 10 s video, <k> was calculated for each frame using the traditional speckle contrast algorithm based on convolution with a square ones matrix (<k>) (Equation 1), and the modified speckle contrast algorithm based on convolution with a square identity matrix (<k′>_{Identity})) (Equation 6). In each calculation, the full 320×240 image size was used to define a full-image size <k>_{FULL}. In addition, a ½ cropped 160×120 image and ¼ cropped 80×60 image was used to extract half-image (<k>_{HALF}) and quarter-image (<k>_{QUARTER}) size speckle crop indexes, respectively. These full-size and cropped image approaches were applied to both <k> and <k′>. Finally, the average value across all frames was calculated using the full 100 fps video stream <k>_{FPS_100}, as well as with a tenfold temporally down-sampled (effective 10 fps) video stream <k>_{FPS_010}. Taken together, there were 12 different <k> values calculated for each 10s video stream based on type of convolution, type of image cropping, and type of effective frame rate used for data processing (FIG. 8). These defined 12 different approaches to calculating LSFI values for each 10s video per Equation 7: LSFI_{Ones,FULL,FPS_100}, LSFI_{Ones,HALF,FPS_100}, LSFI_{Ones, QUARTER, FPS_100}, LSFI_{Ones,FULL,FPS_010}, LSFI_{Ones,HALF,FPS_010}, LSFI_{Ones,QUARTER,FPS_010}, LSFI_{Identity,FULL,FPS_100}, LSFI _{dentity,HALF,FPS_100}, LSFI_{Identity,QUARTER,FPS_100}, LSFI_{Identity,FULL,FPS_010}, LSFI_{Identity,HALF,FPS_010}, and LSFI_{Identity,QUARTER,FPS_010.}

The noninvasively derived LSFI signal maintained a steady baseline for 15 minutes prior to blood draw, decreased with decreasing blood volume, and increased with addition of crystalloid (FIG. 7). Between 90 and 115 minutes the speckle sensor was misaligned, resulting in an erroneous LSFI signal (gray portion in FIG. 7). The shape of the LSFI signal over time remained consistent regardless of type of convolution, degree of image cropping, or effective frame rate (FIG. 8), and a cross correlation of the 12 variations of LSFI against each other yielded a correlation coefficient R of 0.99-1.00, indicating that significant reductions in processing burden can be achieved (by decreasing frame rate, analyzing a cropped image, and averaging across a diagonal square matrix rather than a full square matrix), without compromising LSFI signal integrity.

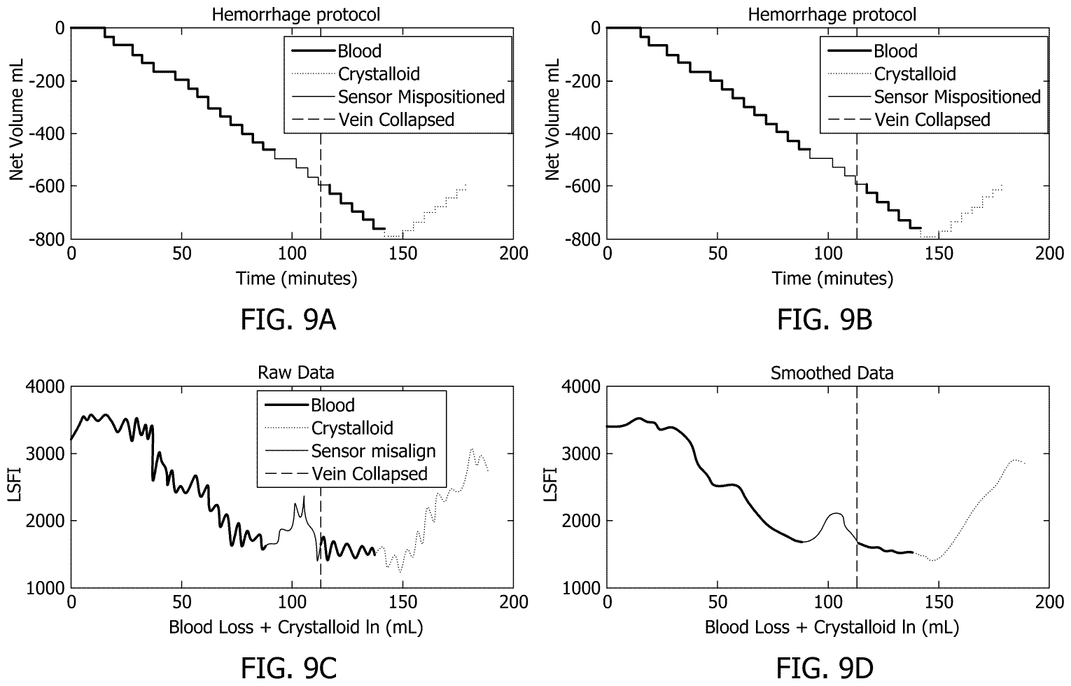

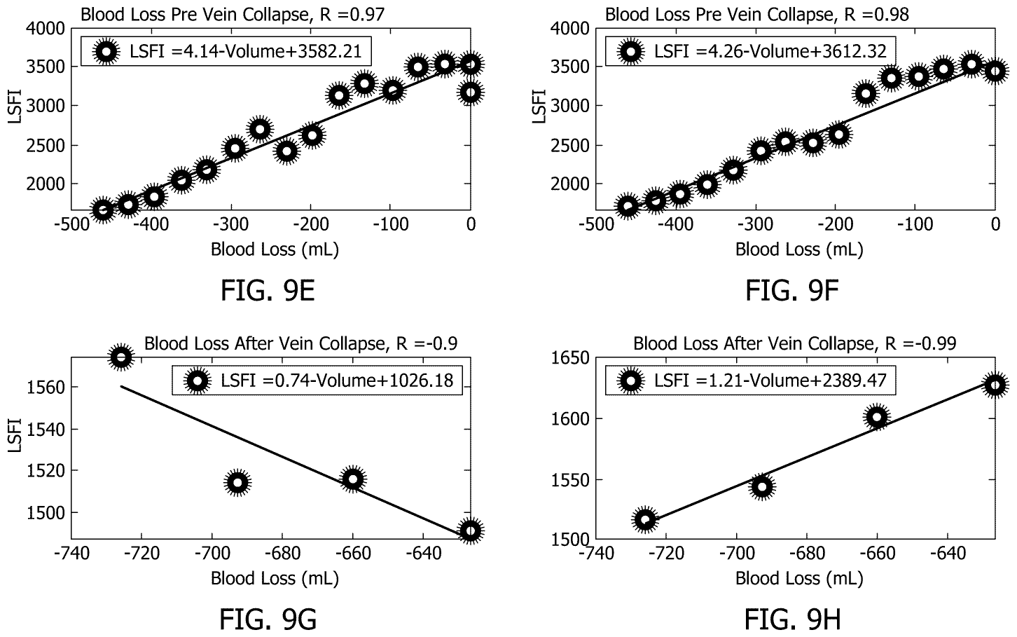

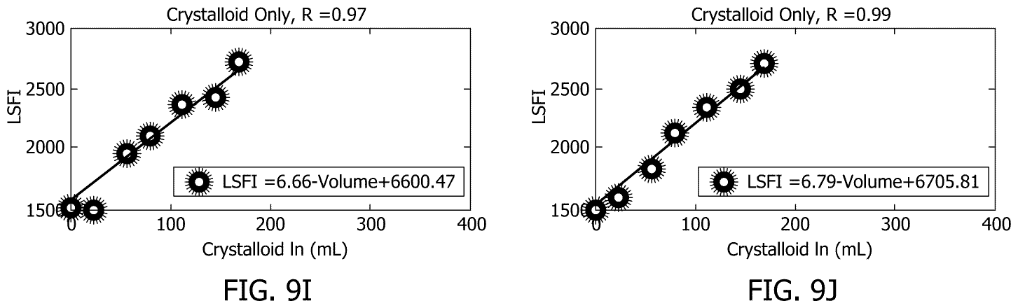

A linear regression between unsmoothed and smoothed (10 point moving average) LSFI signal and net fluid volume was determined for each LSFI variation (FIG. 9). LSFI showed a strong linear correlation with fluid volume change during the entire experiment (Pearson R=0.88). However, the regression between LSFI and fluid volume change during the first 2 hours, where blood volume decreased, was different from the regression between LSFI and fluid volume change in the last 35 minutes, where blood volume was fixed and crystalloid was added. Specifically, the regression between LSFI_{Ones,FULL,FPS_100} and blood loss volume prior to vein collapse showed an R=0.97 in unsmoothed data and R=0.98 in smoothed data, with a slope of 4.14/mL and 4.24/mL, respectively (FIGS. 9E and 9F). During blood loss post-vein collapse, the regression coefficient between LSFI_{Ones,FULL,FPS_100} and blood loss was R=−0.9 in unsmoothed data and R=0.99 in smoothed data, with a slope of −0.74/mL and 1.21/mL, respectively (FIGS. 9G and 9H). Meanwhile, the regression between LSFI_{Ones,FULL,FPS_100} and crystalloid input volume was R=. 97 in unsmoothed data and R=0.99 in smoothed data, with a slope of 6.66/mL and 6.79/mL, respectively (FIGS. 9I and 9J). Similar strong but distinct correlations for LSFI vs blood loss volume and LSFI vs crystalloid input volume were found with the 11 other variations of LSFI processing.

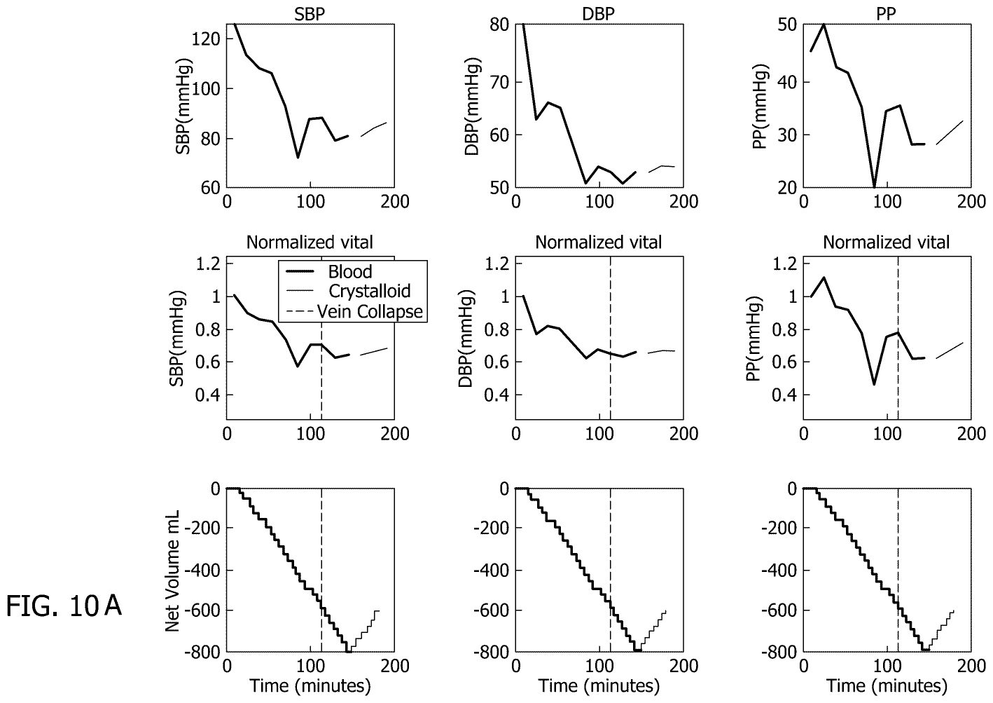

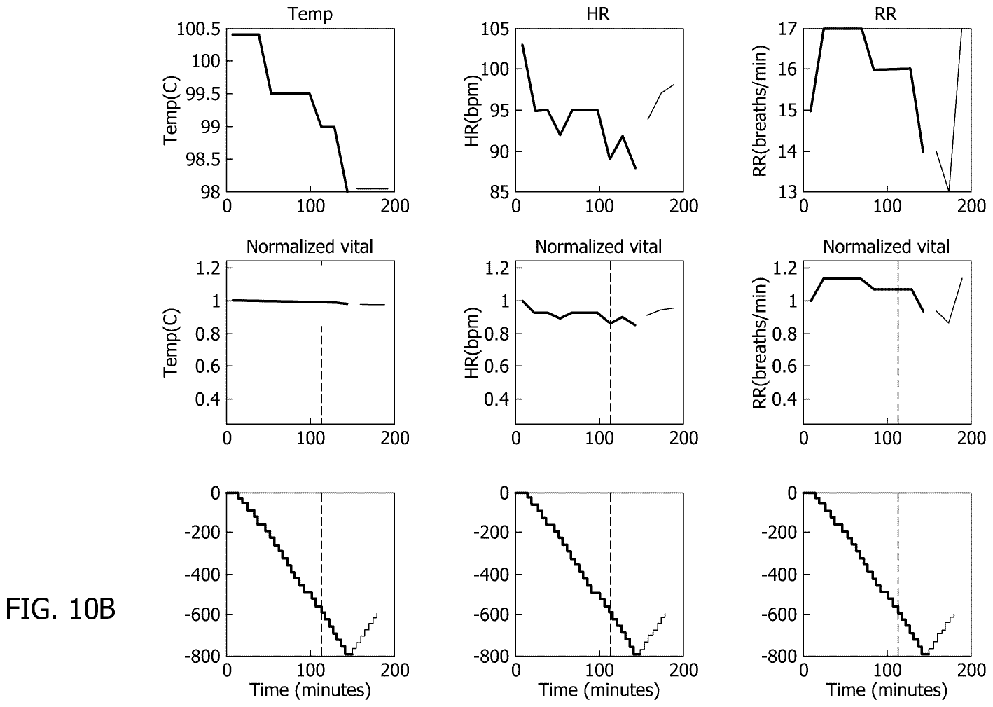

Vital signs, including systolic and diastolic blood pressure, pulse pressure, temperature, heart rate, and respiratory rate were recorded noninvasively at 15 minute intervals, and appeared to show a similar downward trend with blood loss followed by upward trend with administration of crystalloid (FIG. 10). Vital signs were obtained at only 3 time points during crystalloid infusion, making linear correlation between vital signs and crystalloid volume challenging to interpret. The middle panel shows normalized vital signs, where each value was divided by the initial value.

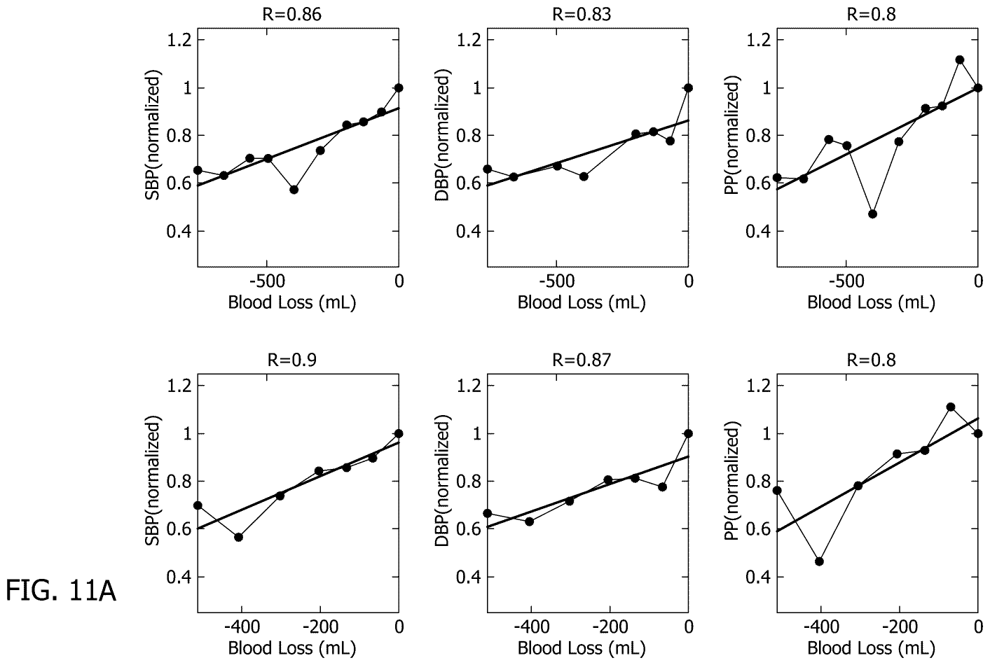

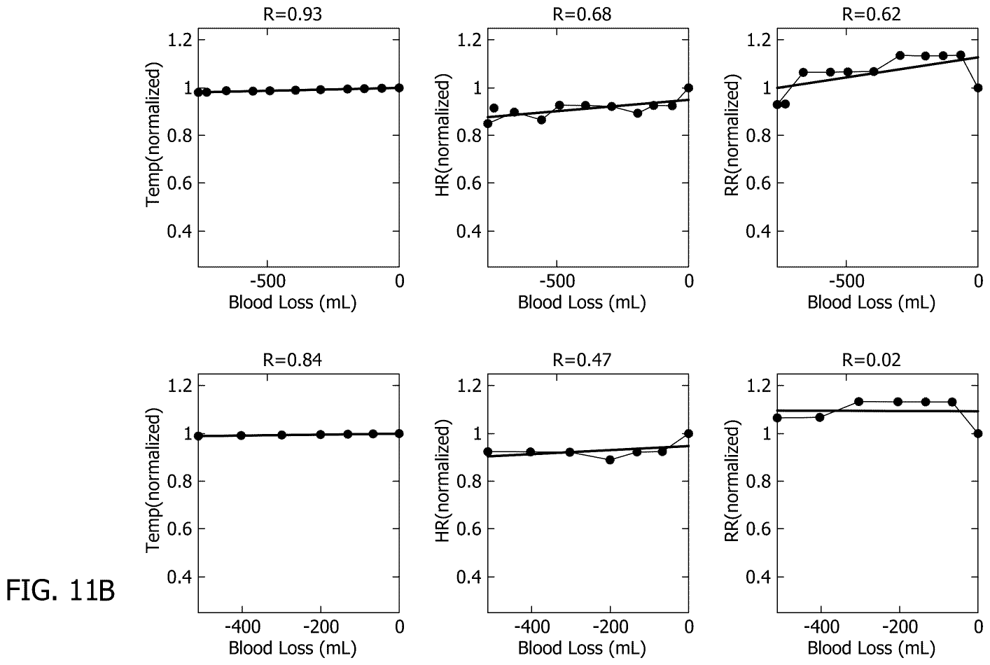

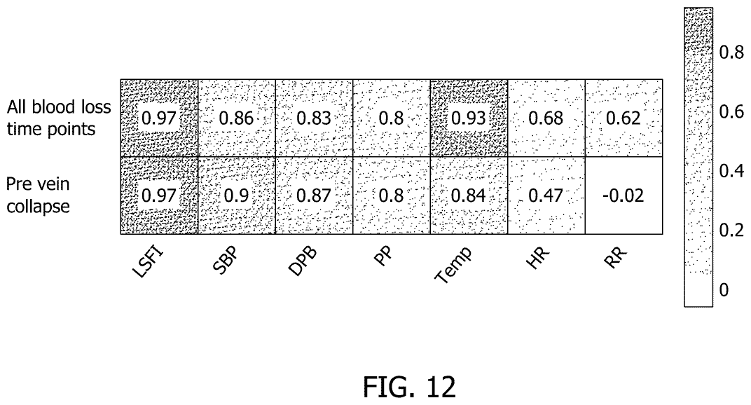

FIG. 11 demonstrates the correlation between volume of blood loss and each measured normalized vital sign, with the top panel including all time points and the bottom panel including times preceding vein collapse and before crystalloid infusion. While body temperature showed the strongest correlation with blood volume loss, unlike LSFI, body temperature showed no increase with crystalloid infusion (FIG. 11). Indeed body temperature may have decreased steadily with time as a result of anesthesia rather than as a result of blood loss. Future studies will include control swine that are anesthetized for the same duration as the hemorrhage protocol to track changes caused by anesthesia over time. LSFI had the highest correlation to blood loss (R=0.97) and crystalloid infusion (R=0.97) of all the vital signs tracked (FIG. 12), underscoring the accuracy and added value of this technique to monitor dynamic changes in peripheral perfusion.

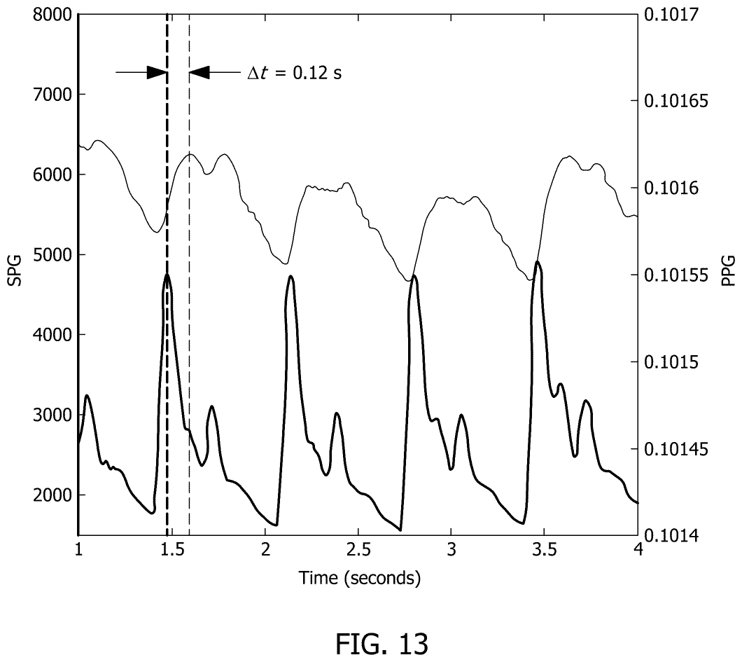

Much information can be extracted from the laser speckle and hemodilution sensors, and this data can be combined in novel algorithms for early detection of hemorrhage, postpartum hemorrhage, treatment response, and general vascular hemodynamic monitoring. For the hemodilution sensor, we anticipate that the ratio of water to hemoglobin will increase as blood loss increases due to water being pulled into the vasculature from the interstitial fluid in the body's attempt to increase circulating blood volume. There will be an AC component and a DC component, similar to pulse oximeters. To extract a vascular hemodilution parameter, we will calculate a ratio of ratios: R=(ACλwater/DCλwater)/(ACλhemoglobin/DCλhemoglobin). Similar to blood oxygen saturation, we will determine the hemodiltion (HD) according to the equation: HD=(k1−k2*R)/(h3−k4)*R, where the k constants are empirically determined for each device during calibration across a variety of hemoglobin concentrations. This sensor can track the effects of various interventions such as fluid supplementation, infusion with packed red blood cells and/or transfusion. This will allow medical providers to identify dangerously low hemoglobin concentrations so they can augment their care to increase hemoglobin concentration. It also has the potential to assess intravascular water content and extravascular water content for edema monitoring during conditions such as preeclampsia. For laser speckle, we can extract laser speckle contrast (k and k′, described above), and calculate the mean, standard deviation, peak-to-peak amplitude, pulse variability, frequency content including pulse rate, pulse rise time and fall time, analysis of the data in the frequency domain, including frequency content and harmonics. This can be compared to standard photoplethysmogaph (PPG) data (which can be obtained by simply taking 1 over the natural log of the mean of the intensity image and plotting this value over time) to extract additional information such as the time differences in peak location measured via LSFI and PPG (FIG. 13).

Diagnosis could be determined by setting a threshold for a single metric or multiparameter index that would indicate hemorrhage, such as a certain % change from baseline levels, reaching a certain slope, identifying a local minimum or maximum in the derivative or second derivative of the time series data. In settings of hemorrhage, we expect the mean amplitude of the LSFI to decrease, and analysis of the rate of LSFI change over time may help us to discern important parameters such as when the patient is still compensating for blood loss or if their veins have collapsed. You will note in FIG. 5 that the LSFI readings plateaued after the vein collapsed, compared with the precipitous decrease prior to vein collapse. This plateau could be an indicator that the patient can no longer compensate and will likely go into hypovolemic shock, for example. Further, the slope of the decrease will likely be an important indicator of the rate of blood loss, as well as the ability of the patient to compensate for blood loss. This also holds true for treatment of blood loss, where we observed a sharp increase in LSFI signal with infusion of crystalloids. It is possible a “compensation challenge” could be performed in patients prior to surgery or labor, either via a mild blood loss or fluid bolus, to identify patients who do not compensate well as these patients could be at higher risk for hypovolemic shock from a relatively small volume of blood loss. Furthermore, more sophisticated algorithms could be developed that incorporate a patient's medical history and variables such as height, weight, BMI, SBP, DBP, PP, mean HR, relevant medications, use of anesthesia and type if applicable, and starting hematocrit or hemoglobin concentration such that the diagnosis algorithms become personalized to each patient for a more accurate determination of early stage hemorrhage as well as treatment monitoring. Once more data is collected, we will employ machine learning techniques to further improve our predictions.

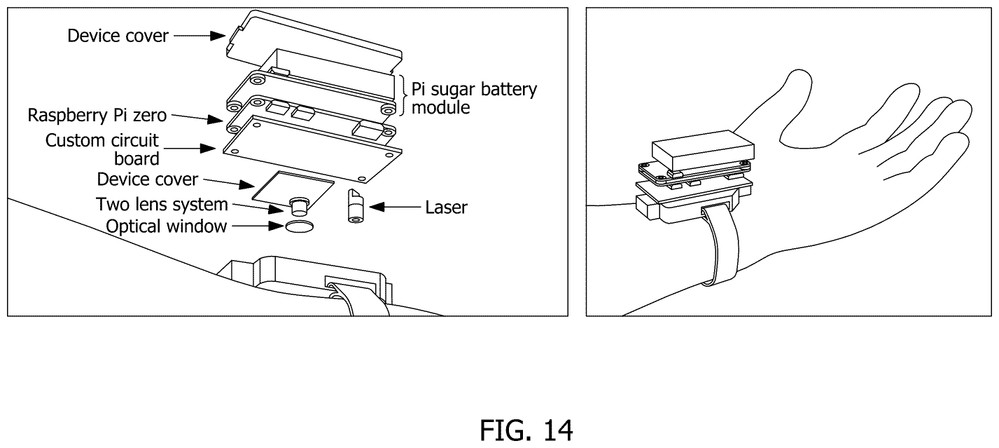

A wearable laser speckle sensor (FIG. 14) uses a reflectance mode design (although could be made in transmission) with the same camera, 2-lens system, optical window, and laser as the design illustrated in FIG. 4. However, this system is fully wearable, wireless, and powered by a Pi sugar battery module connected to a Pi zero 2 W computer board and custom wearable electronics that stabilize the laser output. All components are held in place in a small form factor using custom 3D housings. This new design is showcased in FIG. 14.

The strong correlations observed by our laser speckle sensor in both blood loss and crystalloid infusion demonstrate high sensitivity to peripheral perfusion and compensatory mechanisms to stabilize central hemodynamics. These findings have major implications for the ability of this sensor to detect similar changes in trauma patients, surgery patients, and pregnant women to provide an early alert for dangerous blood loss, as well as provide a method to monitor response to treatment and/or interventions that may affect peripheral perfusion and vascular hemodynamics. Further, this device could be used to assess a given patient's ability to compensate for blood volume loss, a notoriously patient-dependent response. This could help with surgical and labor plans to identify high risk individuals that may not have strong compensatory responses and are thus more susceptible to hypovolemic shock.

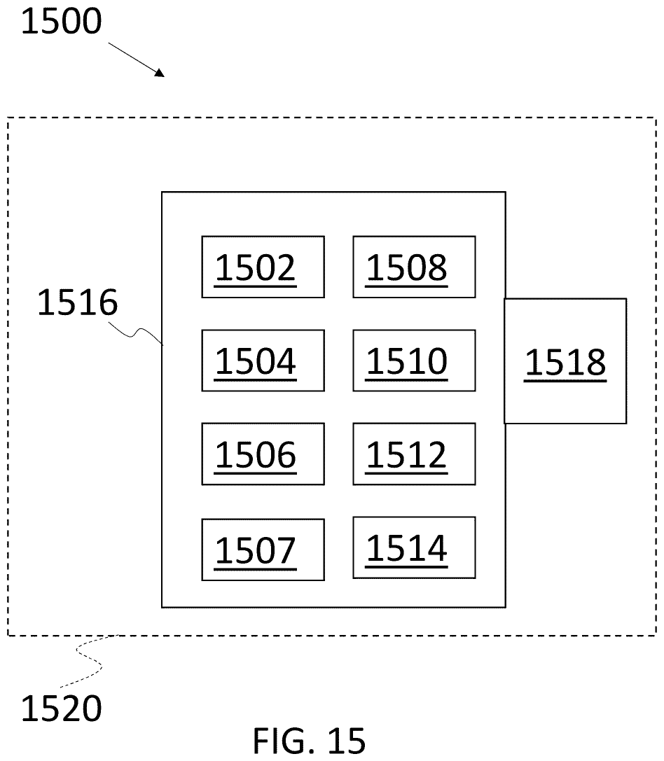

FIG. 15 is a block diagram of an example system 1500 for monitoring peripheral blood flow of a patient using a wearable device similar to the devices described above. The system includes a physiologic sensor 1502 (also referred to sometimes herein as sensor 1502) for monitoring peripheral blood flow of the patient, a processor 1504 communicably coupled to the physiologic sensor, a memory 1506 communicably coupled to the processor, an input device 1507, a display device 1508, a blood flow restriction device 1510, a communication interface 1512, and a power source 1514. A housing 1516 fully or partially covers, encloses, and/or houses the sensor 1502, processor 1504, memory 1506, input device 1507, output device 1508, blood flow restriction device 1510, communication interface 1512, and power source 1514. The system 1500 includes an attachment device 1518 for attaching the housing 1516 and the components within/on the housing to the patient as a wearable device 1520.

The physiologic sensor 1502 is any sensor or sensors operable to monitor the flow of blood of a patient. In the example embodiment, the sensor 1502 includes the laser speckle sensor described above. In other embodiments, the sensor 1502 includes the laser speckle sensor and the hemodilution sensor. In other embodiments, the sensor 1502 includes a photoplethysmography sensor, a pulse oximeter, or any other suitable sensor.

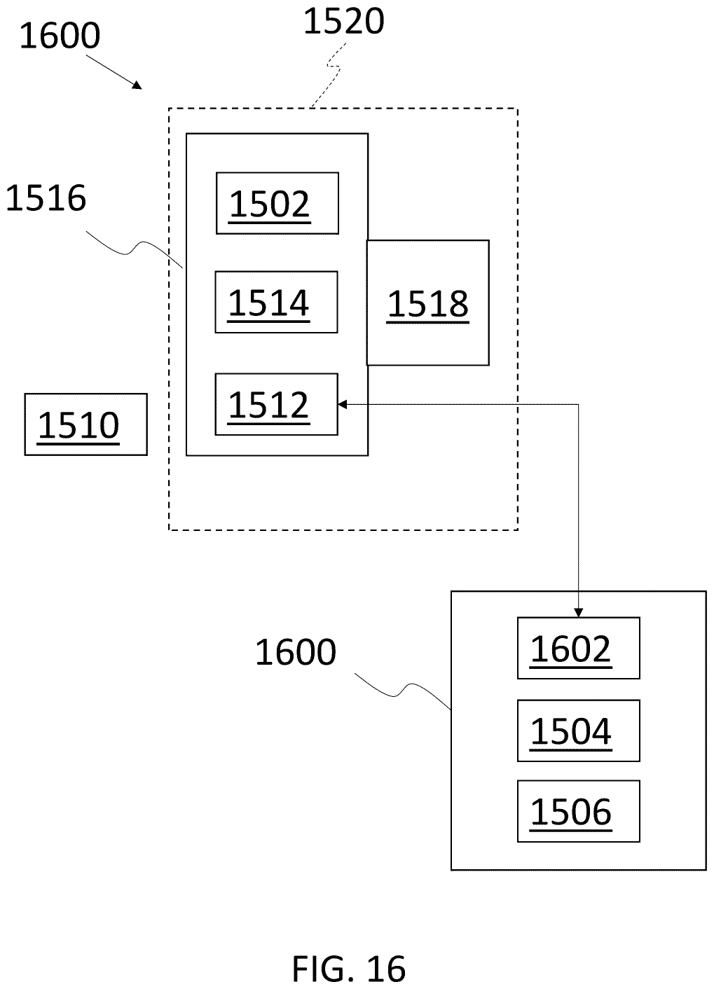

As will be described in more detail below, the processor 1504, acting according to the instructions in the memory 1506, monitors peripheral blood flow of the patient using the sensor 1502. The processor 1504 may be a controller, a computer, a microcontroller, a microcomputer, a programmable logic controller (PLC), an application specific integrated circuit, an integrated circuit, or any other programmable circuits. Additionally, memory 1506 may generally be or include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory 1506 may generally be configured to store suitable computer-readable instructions that, when implemented by the processor 1504, configure or cause processor 1504 to perform various functions described. Although illustrated separately, the processor and the memory may be included in a single component, such as the example computer board discussed above. Moreover, in some other embodiments, such as the embodiment of FIG. 16, the processor and the memory are not included within the housing 1516 of the wearable device 1520.

The input device is any device operable to receive an input from a user. The input device may be, for example, a keyboard or keypad, one or more buttons, dials, or switches, a touchscreen, a light sensor, a pressure sensor, or the like.

The output device 1508 is operable to output information to the patient, a doctor, a nurse, or other suitable party. The output device 1508 may be a display device (e.g., a liquid crystal display (LCD), organic light emitting diode (OLED) display, cathode ray tube (CRT), “electronic ink” display, one or more light emitting diodes (LEDs)) or an audio output device (e.g., a speaker or headphones). Some other embodiments do not include the output device 1508. In some embodiments, the input device and the output device are combined in a touchscreen display device.

The blood flow restriction device 1510 is operable to selectively restrict the patient's blood flow proximal the sensor 1502 as part of a calibration process for the system 1500. The blood flow restriction device 1510 may be a cuff, a tourniquet, a rubber/elastic band, a strap, or any other device suitable for restricting patient blood flow near the sensor 1502. In the example embodiment, the blood flow restriction device 1510 is controllable by the processor 1504. In other embodiments, the blood flow restriction device is selectively operated by a user, such as the patient, a nurse, a doctor, or the like. In still other embodiments, the blood flow restriction device 1510 is a separate component not included within the housing 1516 of the wearable device 1520. Moreover, in some embodiments, the blood flow restriction device is not a part of the system 1500.

The communication interface 1512 enables the wearable device 1520 (and the processor 1504) to communicate with remote devices and systems, such as other/remote sensors, remote computing devices (including tablets and mobile phones), other components of the system, and the like. The communication interface 1512 may be a wired or wireless communications interface that permits the controller to communicate with the remote devices and systems directly or via a network. Wireless communication interfaces may include a radio frequency (RF) transceiver, a Bluetooth® adapter, a Wi-Fi transceiver, a ZigBee® transceiver, an infrared (IR) transceiver, a near field communication (NFC) transceiver, and/or any other device and communication protocol for wireless communication. (Bluetooth is a registered trademark of Bluetooth Special Interest Group of Kirkland, Washington; ZigBee is a registered trademark of the ZigBee Alliance of San Ramon, California.) Wired communication interfaces may use any suitable wired communication protocol for direct communication including, without limitation, USB, RS232, I2C, SPI, analog, and proprietary I/O protocols. In some embodiments, the wired communication interface 1512 may include a wired network adapter allowing the computing device to be coupled to a network, such as the Internet, a local area network (LAN), a wide area network (WAN), a mesh network, and/or any other network to communicate with remote devices and systems via the network.

The power source 1514 provides power for operation of wearable device 1520 and its components. In the example embodiment, the power source 1514 is a battery. The battery may be a primary or secondary battery of any suitable chemistry. In other embodiments, the power source 1514 is one or more capacitors, photovoltaic cells, or any other suitable source of power for the wearable device 1520. In still other embodiments, the power source 1514 is a power input that receives power from an external power source, such as an external AC or DC power supply, AC mains power, an external battery, an external uninterruptible power supply (UPS), or the like.

In the example embodiment, the housing 1516 encloses the sensor 1502, processor 1504, memory 1506, communication interface 1512, and power source 1514, while the output device 1508 and the flow restriction device 1510 are not fully enclosed within the housing. In some other embodiments, the output device (particularly when the output device is an audio output device) is fully enclosed within the housing. The housing protects the components within the housing and packages them into a wearable format. The attachment device 1518 is coupled to the housing and is used to attach the housing (and thereby the wearable device 1520) to the patient. The attachment device may include an elastic strap, one or more bands with appropriate connections (e.g., buttons, snaps, hook/loop, pins/holes, etc.), clips, clamps, or the like. Although described separately, the attachment device may be a part of or integrally formed with the housing in some embodiments.

FIG. 16 is a block diagram of another example system 1600 for monitoring peripheral blood flow of a patient using a wearable device similar to the devices described above. Common components with the system 1500 are identified by the same reference numbers and operate similarly unless otherwise described. In this example embodiment, the processor 1504 and the memory 1506 are not included within the housing 1516 of the wearable device 1520. Rather, the system includes a remote device 1600 including the processor and the memory. The remote device may be an off the shelf computing device such as a computer (including a laptop or a desktop computer), a tablet, a mobile phone, a PDA, a virtual/augmented reality display system, or the like. In other embodiments, the remote device is a specialty device, such as a computing device specifically designed for use in the system 1600. The remote device includes a communication interface 1602 of a type compatible with the communication interface 1512 of the wearable device. In this example, the processor 1504 still monitors the peripheral blood flow of the patient using the sensor 1502, but does so remotely from the wearable device 1520.

In system 1600, the blood flow restriction device 1510 is not included as part of the wearable device 1520. Rather, the blood flow restriction device is external and separate from the wearable device. In some embodiments, the external blood flow restriction device is communicatively coupled to the wearable device 1520 and/or the remote device 1600 to allow the processor 1504 to control the blood flow restriction device and/or to allow the blood flow restriction device to communicate data to the wearable device and/or the processor.

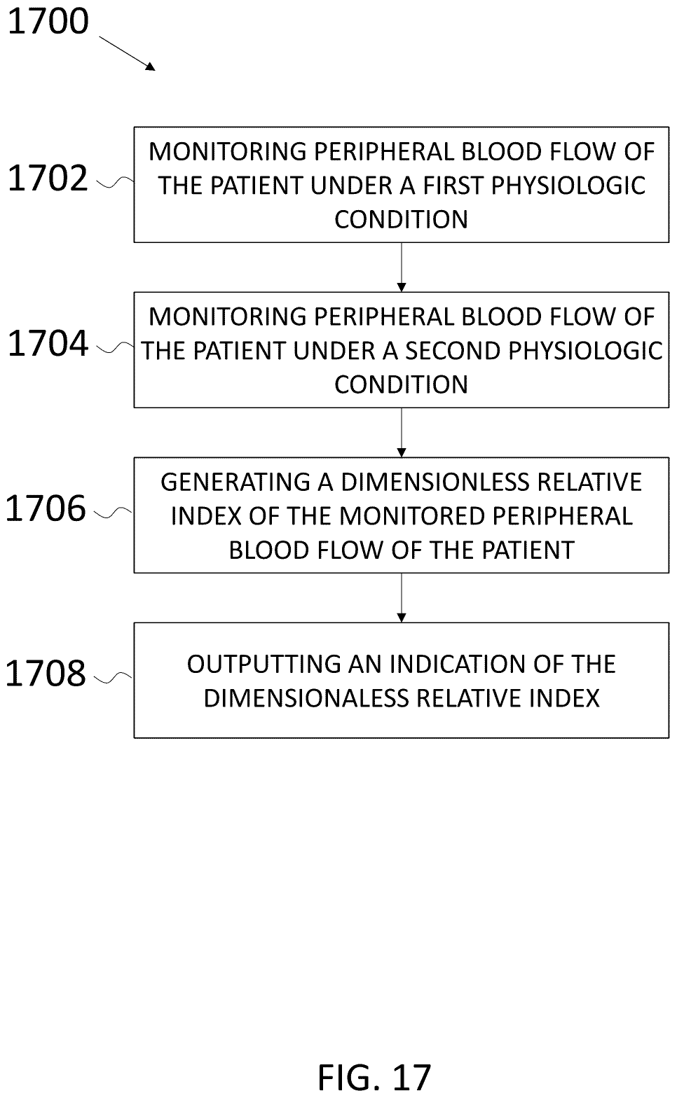

FIG. 17 is a flow diagram of an example method 1700 for monitoring peripheral blood flow of a patient using a wearable device, such as the wearable device 1520. While the method will be described with reference to systems 1500, 1600 and wearable device 1520, the method may be performed using any other system/device described herein, or using any other suitable wearable device or system.

At 1702, the peripheral blood flow of the patient is monitored using the wearable device 1502 under a first physiologic condition. In the example embodiment, the first physiologic condition is an artificially restricted blood flow. In some embodiments, the first physiologic condition is an unrestricted blood flow. In the example embodiment, the blood flow restriction device 1510 is used proximal the sensor 1502 to artificially restrict the blood flow that is sensed by the sensor 1502. In the example embodiment, the blood flow restriction device 1510 is not part of the wearable device 1502, though it is included in the wearable device in some other embodiments.

In the example embodiment, a person or other device controls the blood flow restriction device 1510 to start and end the first physiologic condition. The processor 1504 is programmed to receive a first indication that conveys that the first physiologic condition is starting (i.e., that the blood flow restriction device is now being used to artificially restrict blood flow) and a second indication that conveys that the first physiologic condition is ending (i.e., that the blood flow restriction device is no longer being used to artificially restrict blood flow). The second indication is generally also an indication that the second physiologic condition is beginning. The first and second indications may be electronic indications received from another device controlling the blood flow restriction device, a manual indication by the person operating the blood flow restriction device (such as by using the input device 1507 on the wearable device).

In some embodiments, the processor 1504 controls the blood flow restriction device to start and end the first physiologic condition. The processor may operate the blood flow restriction device directly or indirectly. Thus, even in embodiments in which the processor controls the blood flow restriction device, the processor may receive an indication that the to restrict the patient's blood flow may receive an indication that the first physiologic condition is starting (i.e., that the blood flow restriction device is now being used to artificially restrict blood flow) and a second indication that conveys that the first physiologic condition is ending (i.e., that the blood flow restriction device is no longer being used to artificially restrict blood flow) from the device through which the processor controls the blood flow restriction device or may determine the indications based on when the blood flow restriction device is fully restricting the blood flow or has fully ceased restricting the blood flow.

In the example embodiment, monitoring the patient's peripheral blood flow (under any physiologic conditions) using the wearable device 1520 includes determining numerical values representing the blood flow as described above. Thus, monitoring peripheral blood flow of the patient under the first physiologic condition includes determining a first numerical value representing the peripheral blood flow of the patient under the first physiologic condition.

As will be explained further below, measurement of the peripheral blood flow during the artificial restriction of the peripheral blood flow is performed to provide a baseline that is used for generating a dimensionless relative index of the unrestricted monitored blood flow. Thus, a single measurement is typically desired to represent the restricted peripheral blood flow. However, in various embodiments, the processor 1504 may take one measurement of the blood flow while the artificially restricted blood flow persists or may take multiple measurements. When a single measurement is taken, it is preferably taken some time after the restriction begins and before the end of the time period that the condition persists to represent capture of a steady state measurement. For multiple measurements, any suitable mathematical/statistical techniques may be used on the multiple measurements to determine a single measurement. For example, the multiple measurements may all be averaged, the median or mode of the measurements may be used, the high and low measurements may be discarded and the remaining measurements may be averaged, all of the measurements within a certain window of time may be averaged, etc.

At 1704, the peripheral blood flow of the patient is monitored using the wearable device 1502 under the second physiologic condition. In the example embodiment, the second physiologic condition is an unrestricted blood flow (i.e., the patient's current blood flow without any artificial modifications/restrictions applied). In some other embodiments, the first physiologic condition is an unrestricted blood flow and the second physiologic condition is an artificially restricted blood flow. In the example embodiment, monitoring peripheral blood flow of the patient under the second physiologic condition includes determining a second numerical value representing the peripheral blood flow of the patient under the second physiologic condition.

The monitoring of the peripheral unrestricted blood flow of the patient (whether it is the first physiologic condition or the second physiologic condition) is the normal monitoring of the patient's peripheral blood flow. That is, when the system 1500 is being used to monitor peripheral blood flow of a patient, it is the unrestricted peripheral blood flow of the patient that is desired to be monitored (to detect possible PPH, for example). As such, multiple measurements will be acquired while the unrestricted blood flow physiologic condition exists. Unlike the measurements during the artificially restricted blood flow, multiple measurements of the unrestricted blood flow (e.g., all of the measurements, every other measurement, every fifth measurement, each average of five successive measurements, or the like) are kept and individually used.

In the example embodiment, a person or other device controls the blood flow restriction device 1510 to start the second physiologic condition (and end the first physiologic condition). The processor 1504 is programmed to receive the second indication that conveys that the first physiologic condition is ending (i.e., that the blood flow restriction device is no longer being used to artificially restrict blood flow) and the second physiologic condition is starting. In some embodiments, the second indication may convey only that the first physiologic condition is ending and a third indication is received to convey that the second physiologic condition is beginning. The third indication may be of the same type of indication as the first and second indication or may be any other suitable type of indication.

A dimensionless relative index (“DRI”) of the monitored peripheral blood flow of the patient is generated at 1706. The DRI is an index of the patient's monitored blood flow without artificial restriction based on the monitored artificially restricted blood flow. Thus, depending on which physiologic conditions are the first and second physiologic conditions, the DRI may index the patient's blood flow under the second physiologic condition based on the monitored peripheral blood flow under the first physiologic condition or may index the patient's blood flow under the first physiologic condition based on the monitored peripheral blood flow under the second physiologic condition. Either way, the monitored restricted blood flow is being used as a baseline/low bound of blood flow in the patient and a dimensionless index of the monitored unrestricted blood flow is generated relative to that baseline/low bound. Broadly, and without limiting the generation of the DRI to a particular mathematical definition of the term, the generating the dimensionless index is a process of normalizing the monitored, unrestricted blood flow of the patient based at least in part on the artificially restricted blood flow.

In some embodiments, the DRI is generated for multiple numerical values of the unrestricted blood flow. That is, each time a numerical value representing the unrestricted blood flow is determined, the DRI is determined for that value based on the numerical value of the artificially restricted blood flow.

To generate the DRI of the monitored peripheral blood flow of the patient, a mathematical/statistical operation is performed using the first numerical value and the second numerical value representing the blood flow during the first and second physiologic conditions. The following examples of how the DRI may be determined will be described for an embodiment in which the first physiologic condition is the artificially restricted blood flow and the second physiologic condition is the unrestricted blood flow, but the same techniques may be modified as appropriate for use in embodiments in which the physiologic conditions are reversed.

In the example embodiment, the DRI is generated by dividing the second numerical value by the first numerical value.

In other embodiments, a highest peripheral blood flow of the patient is determined (for example, by selecting a highest value recorded while the unrestricted blood flow was being monitored or from previous measurements of the same patient during an unrestricted blood flow condition) and set as a maximum bound. The first numerical value is set as a minimum bound. The DRI is generated by rescaling the second numerical value relative to the set maximum and minimum bounds. In some embodiments, the maximum bound is assigned a value of one, the minimum bound is assigned a value of zero, and the second numerical value is rescaled to a value between zero and one. In other embodiments, the minimum and the maximum bounds may be assigned any other suitable values for the creation of the DRI.

In some embodiments, the maximum and or the minimum bounds may be updated based on new blood flow measurements. For example, in some embodiments, if a numerical value greater than the numerical value of the highest peripheral blood flow is determined for the unrestricted blood flow, that new numerical value may be set as a new maximum bound and subsequent numerical values of the unrestricted blood flow are rescaled to between the minimum bound and the new maximum bound.

In other embodiments, any other suitable normalization or other techniques may be used to generate a dimensionless relative index from the first and second numerical values.

At 1708, an indication of the DRI is output by the processor 1504. The output may be a human cognizable output, such as a visual display on a display output device 1508 or an audible output through an audio output device 1508. Additionally or alternatively, the output may be an electronic/digital output. For example, an indication of the DRI may be output to a memory (e.g., memory 1506) for storage or may be output to a remote device for storage or display. The indication of the DRI may be the determined, numerical value of the DRI or may be a relative indication. In some embodiments for example, the value of the DRI may be compared to a threshold value representing low blood flow and the indication of the DRI may indicate whether the DRI is above or below the threshold (e.g., a green color output or an acceptable condition output if the DRI is greater than the threshold and a red color output or a warning/alarm output if the DRI is less than the threshold). This may be combined with or instead of outputting the actual value of the DRI (though the actual value of the DRI is typically output for storage in most cases regardless of any other output or lack thereof). Thus, for example, the value of the DRI may be stored in memory 1506 and output for display on a display output 1508 and that value may be colored green on the display if the DRI is greater than the threshold or red if the DRI is less than the threshold. Similarly, the value of the DRI may be stored in memory and output for display on a display output 1508 and a warning buzzer may be sounded using an audio output device 1508 if the DRI is less than the threshold.

By monitoring blood flow of a patient and generating a DRI as described above, the systems, device, and methods of the present disclosure provide for a measurement of blood flow that is personalized to the particular patient whose blood flow is being monitored. The relative blood flow is not being measured relative to some blood flow averaged or otherwise determined from one or more other patients who are not the current patient and who may have greater or lesser blood flow capacity. Instead, the artificially restricted peripheral blood flow of the actual patient is used as a baseline and measurements of blood flow in the patient are then normalized relative to the patient's actual blood flow. As a result, a more accurate representation of the patient's actual blood flow is produced, which may allow abnormal blood flow (such as during PPH) to be detected earlier and/or more accurately than some known systems. Moreover, these devices, systems, and methods result in a skin tone agnostic measurements of blood flow. Skin tone can affect at least some blood flow measuring systems. In the example embodiment, because any difference in the measurements that would be caused by differences in skin tone appear in both the baseline measurements and the measurements with unrestricted blood flow, determining a DRI using both measurements from the same patient eliminates error that would be caused if, for example, the baseline was from a patient with a different skin tone or an average from patients with different skin tones.

The following experiments were performed using the devices and techniques described above. The experiments will be discussed with respect to the system 1500 for simplicity, but they are applicable to any suitable blood flow monitoring device according to the present disclosure. The system 1500 and/or wearable device 1520 may sometimes be referred to herein or in the figures as the aRMOR device and its output as aRMOR signals.

In these examples, the measurement of blood flow by the sensor 1502 (as processed by the processor 1504 is mean speckle plethysmography (mSPG). The mean SPG (mSPG) is generally presented as a relative measurement because absolute measures of flow require expensive, bulky equipment and complicated processing. Establishing a personalized minimal perfusion benchmark as described above will help determine how close a patient is to reaching their personal peripheral vasoconstriction limit.

Performing an occlusion study at the beginning of data capture helps determine this compensation limit (mSPGocc) while maintaining the advantages of the system 1500: low-cost, small, and simple. This approach may aid clinical decision making by providing an occlusion-normalized index (mSPGocc nom) for calculation of a patient-specific “danger zone”.

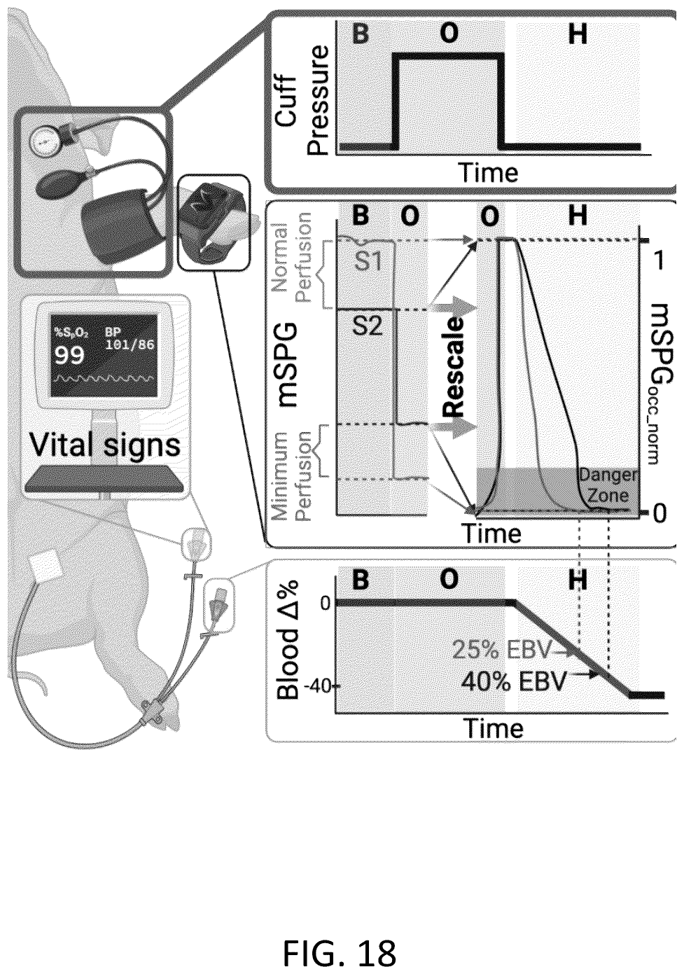

Experimental Design & Methods: With reference to FIG. 18, pneumatic cuff occlusion can be performed prior to a fixed volume hemorrhage in six swine. Swine are used due to similar size and hemodynamic responses to hemorrhage as humans. This experiment will test whether mSPG values during occlusion (mSPGocc) resemble the lowest mSPG values obtained during hemorrhage. Based on prior studies, it is expected that swine have different compensation capacities. Therefore, mSPG values will be normalized by rescaling mSPG obtained at baseline (mSPGₜ₌₀) to 1 and mSPGocc to 0, such that [mSPG_{norm_occ}(t)=(mSPG(t)−mSPG_{occ})/(mSPGₜ₌₀−mSPG_{occ})] (i.e., a DRI is generated).

After scaling, swine will be hemorrhaged to 40% of their estimated blood volume (EBV) over one hour to surpass their peripheral vasoconstriction capacity. FIG. 18 shows the effect of rescaling in swine who have maximum compensation at 25% (S1) or 40% (S2) EBV loss, and the ability to determine “danger zones” in both despite different compensation capacities. A sample size of six swine was chosen in this pilot study because high contrast differences pre- and post-occlusion have been demonstrated using SPG devices. N=6 will enable quantification of how closely the occlusion signals resemble the maximum vasoconstriction state across swine while minimizing the number of swine used. Data is captured continuously by placing the wearable device 1520 on the swine “wrist” starting at swine throughout occlusion (total duration of 5 min) and hemorrhage, until one-hour post-blood draw.

Data Analysis: mSPG processing will be performed on the wearable device 1520 data acquired throughout each swine study. The mSPG signal acquired during pneumatic cuff occlusion (mSPG_{occ}) will be compared to mSPG signal acquired at the most severe stage of the hemorrhage procedure (−40% EBV removed). It is believed that the lowest mSPG signal acquired during hemorrhage, which captures perfusion at maximum peripheral vasoconstriction, will fall within the 80% confidence interval of mSPG_{occ}, therefore supporting the use of mSPG_{norm_occ} to assess PPH risk.

This experiment demonstrates that a subject-specific perfusion floor (mSPG_{occ}) that is reflective of full compensation during severe hemorrhage may be established, enabling improved personalized hemorrhage risk assessment.

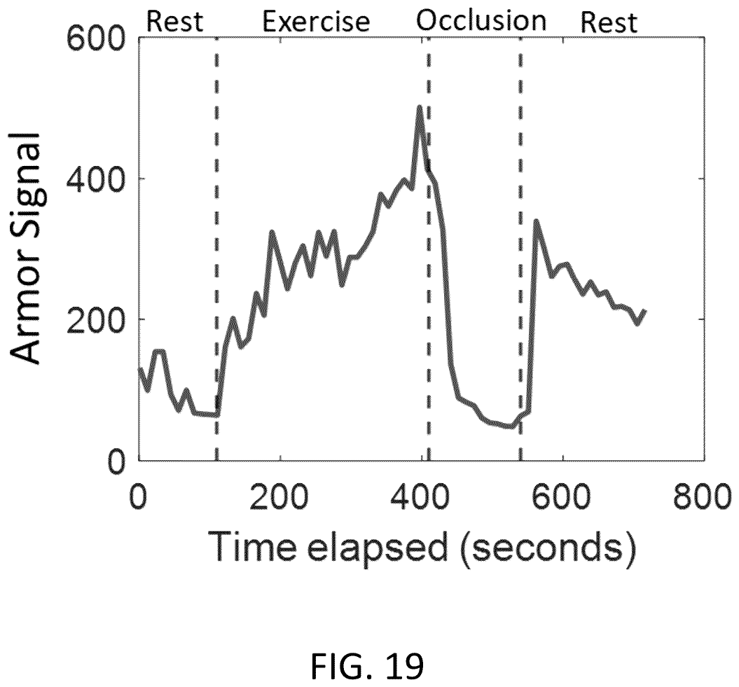

With reference now to FIGS. 19-21, a prototype of the system 1500 was tested on healthy human volunteers. During the human subject experiments, volunteers wore the device on their left wrist to measure changes in hemodynamics. An arm occlusion challenge was used to induce low flow during occlusion and high flow when pressure was released. Cold stimulation on the device arm and 5-minutes of moderate exercise via exercise bike were used to induce hemodynamic changes.

Study participants were divided into two groups. Group 1 participants (n=2) underwent arm occlusion followed by exercise. Group 2 participants (n=4) underwent arm occlusion, cold stimulation, and exercise. It is hypothesized that cold stimulation will cause peripheral vasoconstriction and impact the magnitude of the hemodynamic changes caused by occlusion and exercise.

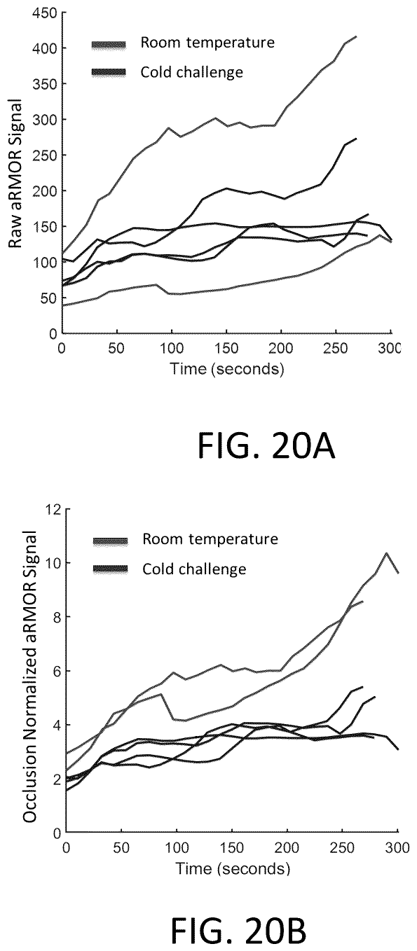

FIG. 19 shows a representative trace from one participant in Group 1. The system 1500 consistently monitored changes in hemodynamics in all subjects, including both increases and decreases in blood flow caused by occlusion Arm occlusion resulted in a minimum aRMOR signal in all subjects and this value was used to normalize data across subjects. FIGS. 20A and 20B show changes in the raw and occlusion-normalized aRMOR signal during exercise. Group 1 and 2 study participants are shown as red and blue traces, respectively

Differences between Groups 1 and 2 are difficult to observe in the raw data (FIG. 20A) but separate significantly in the occlusion-normalized dataset (FIG. 2B), highlighting the utility of the occlusion normalization strategy. Additional analysis of the occlusion-normalized dataset occlusion normalization strategy. Additional analysis of the occlusion-normalized dataset demonstrates that cold stimulation alone did not cause changes from baseline larger than the variance of the signal. However, the rate of increase of the normalized aRMOR signal over the 5-minute exercise period was 3 times larger in Group 1 (μ=0.021, σσ2=4.5e−06) compared to Group 2 (μ=0.007, σσ2=9.2e−06). Although cold stimulation alone did not appear to cause vasoconstriction in the wrist, it does appear to impact the rate at which perfusion increased during exercise.

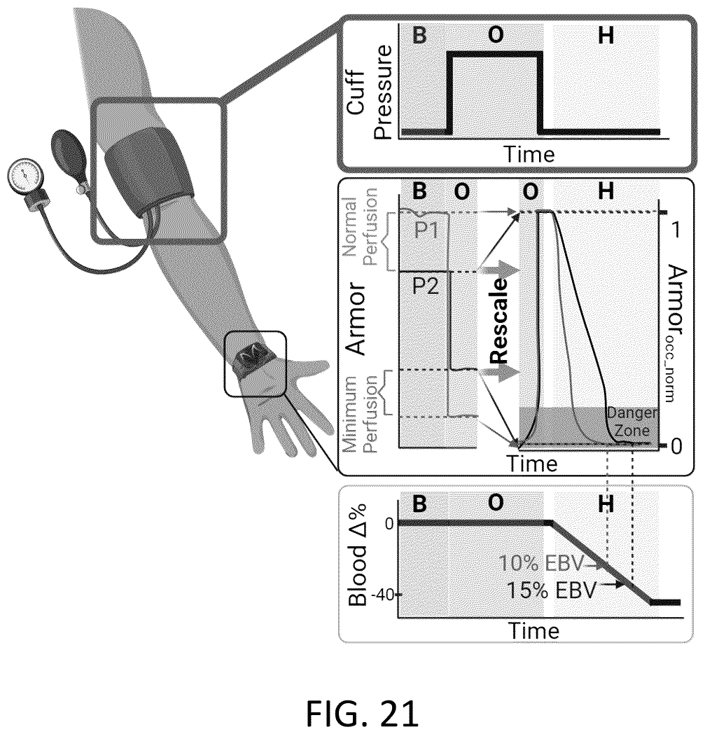

These physiologic challenges provide opportunities to quantify the magnitude of hemodynamic changes that the system 1500 can detect in both high and low perfusion states, similar to those that would be expected during labor and postpartum hemorrhage. It also provides an opportunity to demonstrate the occlusion-normalization strategy's ability to calibrate findings across patients. Laser speckle-based signals are typically presented as relative measurements due to differences in tissue optical properties and the geometry and environment of the system. Establishing a personalized minimal perfusion benchmark will help determine how close a patient is to reaching their peripheral vasoconstriction limit. aRMOR signal values during occlusion are expected to resemble the lowest signal values that will be obtained during hemorrhage, and that the occlusion normalization procedure shown in FIG. 21 and described above will enable future clinical decision support by identifying a patient-specific danger zones during labor and postpartum recovery. Generally, pneumatic cuff occlusion will be performed at the beginning of monitoring blood flow of patients who are laboring or undergoing cesarean delivery. Based on prior studies in swine, it is expected that different patients will have different compensation capacities. The measured aRMOR signal values will be converted to a DRI by rescaling the aRMOR signal obtained at baseline (Armorₜ₌₀) to 1 and aRMOR signal obtained during occlusion (Armor_{occ}) to 0, such that [Armor_{occ_norm}(t)=(Armor(t)−Armor_{occ})/(Armorₜ₌₀−Armor_{occ})]. FIG. 21 shows the effect of rescaling in patients who have maximum compensation at 10% (P1) or 15% (P2) EBV loss, and the ability to determine “danger zones” in both despite different compensation capacities. Moreover, FIG. 21 shows patient-specific “danger-zones” that will be calculated from an occlusion-normalized dimensionless relative index (Armor_{occ_norm}) calibrated to aRMOR values during baseline (B, green) and occlusion (O, orange), to aid data interpretation during hemorrhage (H, yellow). P1 & P2 indicate patient 1 & 2, respectively.

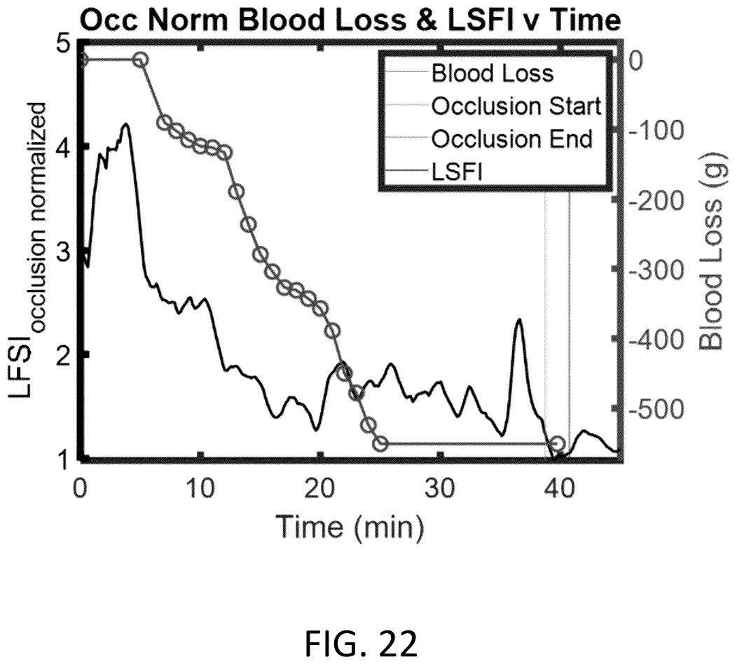

Another experiment testing the wearable device 1520 on adult human blood donors will be described with reference to FIG. 22. A volunteer was seated, and a wearable device 1520 was placed on the left wrist. A blood bag was prepared and placed on a scale. The scale was zeroed prior to the start of blood donation. A 5-minute baseline was performed, and then a research nurse performed phlebotomy on the volunteer's right arm. The weight record from the scale was written down approximately every minute to provide a longitudinal measure of blood loss. Once a weight of 550 g was detected, the phlebotomy procedure was stopped, and the patient rested for 15 minutes. Then a pneumatic cuff occlusion was performed for two minutes to identify the patient's minimum flow, followed by another three-minute rest period.

Data from the wearable device 1520 was processed and the mean laser speckle flow index (LSFI) value per minute was plotted against time as shown in FIG. 22. This data was overlayed with measured net blood loss of the volunteer. The Pearson correlation coefficient from the entire blood draw period was 0.78. However, at around 14 minutes into the blood donation, the LSFI signal plateaued. The signal at the plateau was close to the patient's minimum blood flow value found during pneumatic cuff occlusion, and it is possible that the patient had reached their compensation limit at this point and therefore was not able to vasoconstrict any further. The Pearson correlation coefficient during the time prior to the plateau was 0.956.

The LSFI data from the wearable device 1520 was processed to generate a DRI. In this instance, the LSFI data was divided by the minimum LSFI value recorded during pneumatic cuff, but any other suitable normalization technique could be used. Generating the DRI helped to visualize that the point at which the patient's blood flow stopped decreasing was also when the patient's perfusion was nearing their minimum perfusion state. This demonstrates that the occlusion value is a meaningful measure of perfusion minimum that can be used to identify a perfusion threshold at which patient's may be reaching their physiologic compensation limit. While in this experiment the pneumatic cuff occlusion was performed at the end of the study, occlusion may be performed at the beginning or periodically throughout a study such that the normalized LSFI value can be assessed continuously throughout the study.

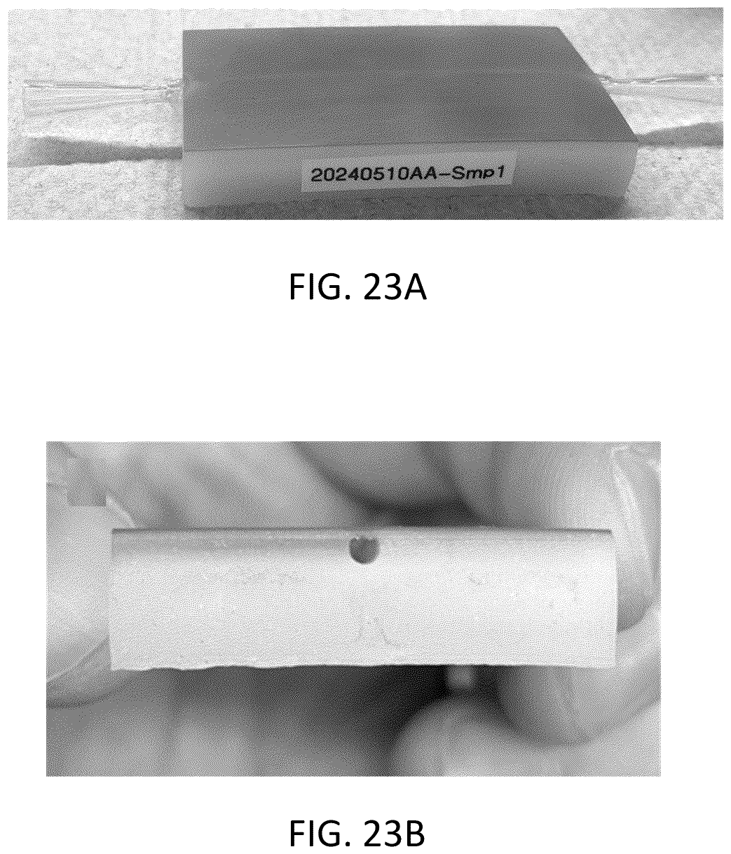

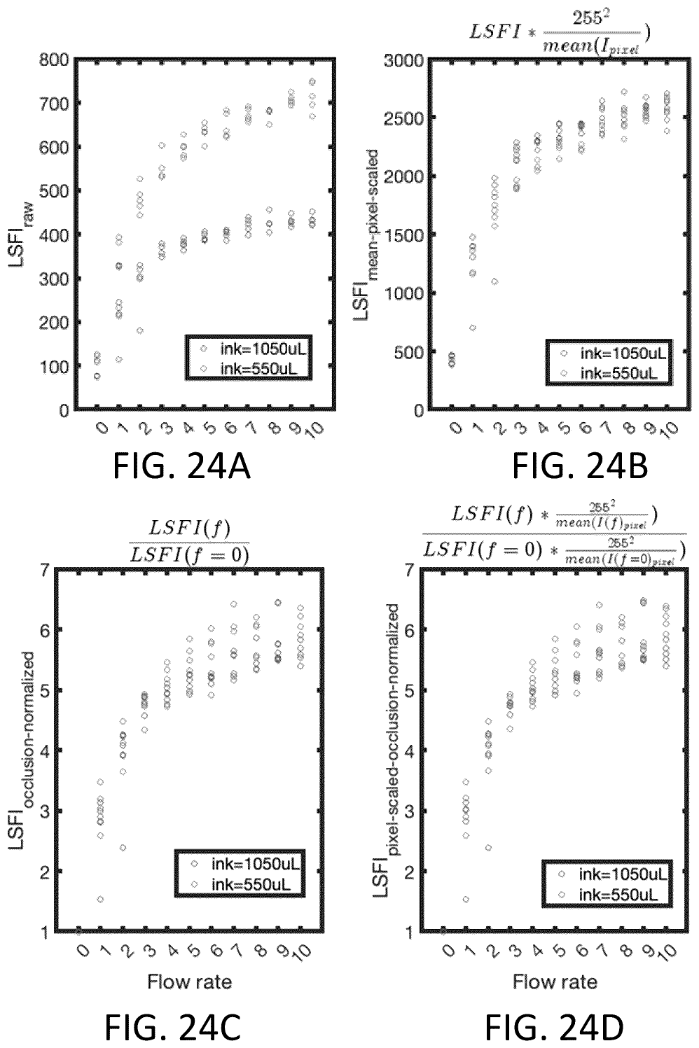

Referring to FIGS. 23A-24D, flow phantom studies designed to test the impact of skin pigmentation on laser speckle perfusion sensor data were performed. The studies used custom-made 3D printed tissue mimicking phantoms made of synthetic resin with varying levels of pigmentation designed to mimic light and dark skin tones in people, one example of which is shown in FIGS. 23A and 23B. Optical scattering in the phantoms was matched to human tissue by adding TiO2 nanoparticles to the resin. Optical absorption of the top layer of the phantom was tuned in the lightly pigmented and darkly pigmented phantom by altering the volume of India Ink added to the resin (550 uL in light vs 1050 uL in dark). India Ink has a similar optical absorption spectrum to melanin and can be dissolved in the resin. The phantoms had a hollow channel near the top of the phantom designed to pass liquid through. The wearable device 1502 was affixed to the phantom with the channel directly beneath the laser and camera. A syringe pump was attached to the flow phantom with tubing and a blood mimicking phantom solution (intralipid, mimics optical scattering of blood) was directed through the tissue mimicking phantoms at flow velocities ranging from 0-10 mm/s. This experiment was performed in triplicate for each flow phantom and the raw LSFI results are shown in FIG. 24A. There is considerable difference between the light and darkly pigmented flow phantoms. FIG. 24B shows a normalization method in which the raw LSFI signal from the wearable device 1502 is multiplied by the maximum sensor intensity (255²=65536) divided by the mean intensity of the pixels in the raw image. This normalization method shows significantly reduced difference as a function of skin pigmentation. FIG. 24C shows the LSFI data normalized by dividing the data by the zero-flow condition, which is physically similar to an occlusion value obtained in living subjects. This normalization method results in a significant reduction in difference as a function of skin pigmentation. In FIG. 24D, another normalization method that combines the image intensity normalization and the zero flow condition normalization also shows a significant reduction in difference in normalized LSFI data in light and darkly pigmented phantoms. This experiment demonstrates that the devices and techniques described in this disclosure provide for accurate blood flow monitoring regardless of skin tone and without requiring any additional adjustment or compensation based on skin tone.

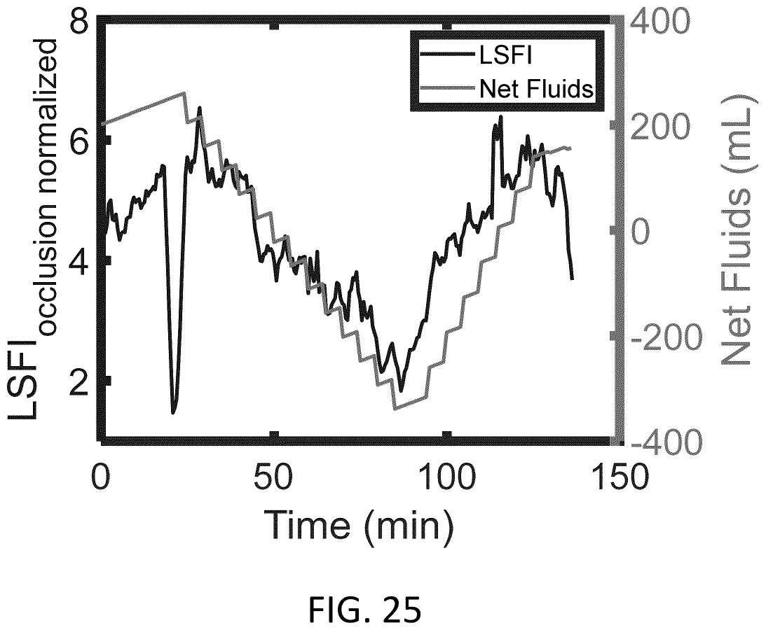

With reference to FIG. 25, another experiment was a swine hemorrhage study in which a female swine was anesthetized, and a wearable device 1502 was placed on the posterior hock of the swine, similar to the wrist of a human. Swine were continuously administered intravenous saline in accordance with standard anesthesia practice. Veterinarians inserted a catheter in the femoral artery to facilitate blood draw. Prior to blood withdrawal, a 20-minute baseline was recorded. After completion of the baseline recording, pneumatic cuff occlusion was performed for 2 minutes, in which a blood pressure cuff was placed proximal to the wearable laser speckle sensor on the swine upper arm and was inflated to 200 mmHg. After the 2-minute occlusion, the pneumatic cuff was deflated and removed from the swine arm. Thereafter, blood withdrawal began at a rate of 3% estimated blood volume every 5 minutes, until reaching 40% EBV. After reaching 40% EBV, there was a 10-minute waiting period followed by resuscitation with intravenous of saline in volumes of 3% EBV every five minutes until half of the blood volume lost was replenished with saline. The changes in net fluids (blood volume removed+intravenous saline injected) and corresponding changes in occlusion normalized LSFI is plotted against time in FIG. 25. The minimum occlusion value has a LSFI value of 1 due to normalization, and the LSFI signal approaches the minimum occlusion value as blood loss becomes severe. This demonstrates once again that the occlusion normalized LSFI value can help determine when hemorrhage is reaching dangerous levels and a patient is reaching their vasoconstriction compensation limit and may soon crash.

The above non-limiting example is provided to further illustrate the present disclosure. It should be appreciated by those of skill in the art that the techniques disclosed in the examples represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A method for monitoring peripheral blood flow of a patient using a wearable device, the method comprising:
monitoring, by the wearable device, peripheral blood flow of the patient under a first physiologic condition;
monitoring, by the wearable device, peripheral blood flow of the patient under a second physiologic condition different than the first physiologic condition;
generating a dimensionless relative index of the monitored peripheral blood flow of the patient:
i) under the second physiologic condition based on the monitored peripheral blood flow under the first physiologic condition; or
ii) under the first physiologic condition based on the monitored peripheral blood flow under the second physiologic condition; and
outputting an indication of the dimensionless relative index.

2. The method of claim 1, wherein:
generating the dimensionless relative index of the monitored peripheral blood flow of the patient comprises generating the dimensionless relative index of the monitored peripheral blood flow of the patient under the second physiologic condition based on the monitored peripheral blood flow under the first physiologic condition;
the first physiologic condition comprises an artificially restricted peripheral blood flow; and
the second physiologic condition comprises peripheral blood flow of the patient without an artificial restriction.

3. The method of claim 2, further comprising:
occluding peripheral blood flow of the patient proximal to the wearable device to create the first physiologic condition; and
ceasing occlusion of the peripheral blood flow of the patient to create the second physiologic condition.

4. The method of claim 3, wherein the wearable device includes a blood flow restriction device configured to occlude peripheral blood flow of the patient.

5. The method of claim 1, further comprising:
receiving a first indication of a start of the first physiologic condition; and
receiving a second indication of an end of the first physiologic condition and a start of the second physiologic condition, wherein monitoring peripheral blood flow of the patient under the first physiologic condition is performed in response to receiving the first indication and monitoring peripheral blood flow of the patient under the second physiologic condition is performed in response to receiving the second indication.

6. The method of claim 1, wherein:
monitoring peripheral blood flow of the patient under the first physiologic condition comprises determining a first numerical value representing the peripheral blood flow of the patient under the first physiologic condition;
monitoring peripheral blood flow of the patient under the second physiologic condition comprises determining a second numerical value representing the peripheral blood flow of the patient under the second physiologic condition; and
generating the dimensionless relative index of the monitored peripheral blood flow of the patient comprises generating the dimensionless relative index of the monitored peripheral blood flow of the patient under the second physiologic condition based on the first numerical value and the second numerical value.

7. The method of claim 6, wherein determining the first numerical value representing the peripheral blood flow of the patient under the first physiologic condition comprises determining a plurality of numerical values and determining the first numerical value as an average of the plurality of numerical values, where each numerical value of the plurality of numerical values represents the peripheral blood flow of the patient at a different time while the first physiologic condition persists.

8. The method of claim 6, wherein generating the dimensionless relative index comprises dividing the second numerical value by the first numerical value.

9. The method of claim 6, wherein generating the dimensionless relative index comprises setting a numerical value representing a highest peripheral blood flow of the patient monitored by the wearable device as a maximum bound, setting the first numerical value as a minimum bound, and rescaling the second numerical value to a value between the minimum bound and the maximum bound.

10. The method of claim 9, wherein generating the dimensionless relative index comprises, when a new numerical value representing peripheral blood flow of the patient higher than the numerical value representing the highest peripheral blood flow is determined, setting the new numerical value as a new maximum bound, and rescaling the second numerical value to between the minimum bound and the new maximum bound.

11. A system for monitoring peripheral blood flow of a patient, the system comprising:
a housing configured for wearable attachment to the patient;
a physiologic sensor for monitoring peripheral blood flow of the patient disposed within the housing;
a processor communicably couplable to the physiologic sensor; and
a memory communicably coupled to the processor, the memory storing instructions that, when executed by the processor, program the processor to:
monitor peripheral blood flow of the patient under a first physiologic condition using the physiologic sensor;
monitor peripheral blood flow of the patient under a second physiologic condition different than the first physiologic condition using the physiologic sensor;
generate a dimensionless relative index of the monitored peripheral blood flow of the patient:
under the second physiologic condition based on the monitored peripheral blood flow under the first physiologic condition; or
under the first physiologic condition based on the monitored peripheral blood flow under the second physiologic condition; and
output an indication of the dimensionless relative index.

12. The system of claim 11, wherein:
the instructions program the processor to generate the dimensionless relative index of the monitored peripheral blood flow of the patient under the second physiologic condition based on the monitored peripheral blood flow under the first physiologic condition;
the first physiologic condition comprises an artificially restricted peripheral blood flow; and
the second physiologic condition comprises peripheral blood flow of the patient without an artificial restriction.

13. The system of claim 12, wherein the instructions program the processor to:
occlude peripheral blood flow of the patient proximal the housing to create the first physiologic condition; and
cease occlusion of the peripheral blood flow of the patient to create the second physiologic condition.

14. The system of claim 13, further comprising a blood flow restriction device controllable by the processor and configured to occlude peripheral blood flow of the patient.

15. The system of claim 11, wherein the instructions program the processor to:
receive a first indication of a start of the first physiologic condition; and
receive a second indication of an end of the first physiologic condition and a start of the second physiologic condition, wherein the instructions program the processor to monitor peripheral blood flow of the patient under the first physiologic condition in response to receiving the first indication and to monitor peripheral blood flow of the patient under the second physiologic condition in response to receiving the second indication.

16. The system of claim 11, wherein the instructions program the processor to:
determine a first numerical value representing the peripheral blood flow of the patient under the first physiologic condition;
determine a second numerical value representing the peripheral blood flow of the patient under the second physiologic condition; and
generate the dimensionless relative index of the monitored peripheral blood flow of the patient under the second physiologic condition based on the first numerical value and the second numerical value.

17. The system of claim 16, wherein the instructions program the processor to determine the first numerical value representing the peripheral blood flow of the patient under the first physiologic condition by determining a plurality of numerical values while the first physiologic condition persists and determining the first numerical value as an average of the plurality of numerical values, where each numerical value of the plurality of numerical values represents the peripheral blood flow of the patient at a different time while the first physiologic condition persists.

18. The system of claim 16, wherein the instructions program the processor to generate the dimensionless relative index by dividing the second numerical value by the first numerical value.

19. The system of claim 16, wherein the instructions program the processor to generate the dimensionless relative index by setting a numerical value representing a highest peripheral blood flow of the patient monitored by the system as a maximum bound, setting the first numerical value as a minimum bound, and rescaling the second numerical value to a value between the minimum bound and the maximum bound.

20. The system of claim 11, wherein the processor and the memory are disposed within the housing.
